(19) United States
(12) Patent Application Publication
(10) Pub. No.: US 2016/0096914 A1
(43) Pub. Date: Apr. 7, 2016

(54) URETHANES, POLYMERS THEREOF, COATING COMPOSITIONS AND THEIR PRODUCTION FROM CYCLIC CARBONATES

(71) Applicant: CYCLICOR AB, Viken (SE)

(72) Inventors: Rajni HATTI-KAUL, Lund (SE); Sang-Hyun PYO, Staffanstorp (SE)

(73) Assignee: CYCLICOR AB, Viken (SE)

(21) Appl. No.: 14/890,488

(22) PCT Filed: May 16, 2014
(86) PCT No.: PCT/SE2014/050606

§ 371 Date: Nov. 11, 2015

Related U.S. Application Data
(60) Provisional application No. 61/969,098, filed on Mar. 22, 2014.
(60) Provisional application No. 61/823,990, filed on May 16, 2013.

(51) Int. Cl. C08G 18/73; C08J 9/00; C08G 18/38; C07C 271/12; C07D 319/06; C07D 317/34; C07C 269/04; C08G 18/83; C09D 175/12

(52) U.S. Cl. CPC C08G 18/73; C08G 18/83; C08J 9/00; C08G 18/3855; C08G 18/3831; C09D 175/12; C07D 319/06; C07D 317/34; C07C 269/04; C07C 271/12; C08J 2375/04; C08J 2375/06; C08J 2375/12

(57) ABSTRACT

The present invention relates to functionalized cyclic carbonates, urethanes and polyurethanes, their methods of production and uses thereof.

### FIELD OF THE INVENTION

The present invention relates to the production of functionalized urethane building blocks, polyurethanes and copolymers from cyclic carbonates which may be functionalized. The invention further relates to the use of said polyurethanes for different applications, e.g. coatings.

### BACKGROUND

Cyclic carbonates have attracted attention in recent years as potential monomers for the production of polyurethanes, polycarbonates and copolymers. Polyurethanes are widely used in foams, seatings, seals, high performance coatings and adhesives. The polymers are also expected to find increasing use in biomedical field due to their features of biodegradability and biocompatibility.

Polyurethanes are currently produced industrially using polyols, such as alkanediols and glycerol, and isocyanate, which is derived from the reaction between an amine and phosgene. Since phosgene and low-molecular weight isocyanates have undesirable toxicological profiles, attempts have been made to develop routes to make polyurethanes from other sources however none of these have yet been commercially established. A demand has now emerged for a reduced use of isocyanate in the production of polyurethanes and copolymers, e.g. isocyanate free polyurethanes, for different applications using more environmentally friendly production processes.

Furthermore the cyclic carbonates and materials obtained from cyclic carbonates are useful building blocks for polymer production, and can be further cross-linked and/or polymerized with various isocyanate compounds.

Attempts have been made to develop routes to make polyurethanes from other sources however none of these have yet been commercially established. One way of reducing or avoiding said toxic raw materials is to produce the polymers by ring-opening polymerization (ROP) of cyclic carbonates.

Synthesis of six-membered cyclic carbonates using lipase B-mediated reaction between trimethylolpropane (TMP) and dialkylcarbonate in a solvent-free medium has been recently reported. It was shown that the lipase catalyses mainly the formation of linear carbonates and their conversion to cyclic carbonates is promoted by increased temperature. It was also shown that the use of hydrophobic solvents in the reaction medium increased the proportion of cyclic carbonates formed. The differential specificity of the lipase for primary, secondary and tertiary alcohol groups in the substrate was investigated to increase the selectivity of the production and yield of six-membered cyclic carbonates from diols and dimethylcarbonate.

The cyclic carbonates were prepared from polyols such as trimethylolpropane (TMP), trimethylolethane, di-TMP, and 1,2-propanediol according to the process in Swedish patent application (No. 1150981-7)). This invention was developed to produce cyclic carbonates with or without using catalysts. Polyol compounds were reacted with dialkylcarbonates such as dimethylcarbonate and diethylcarbonate to produce a corresponding linear and/or cyclic carbonate.

There is a demand to find new materials with improved performances as well as more environmentally friendly materials and to find ways to reduce the use of isoccyanate and phosgene in the production of polyurethanes and copolymers.

### SUMMARY

The present invention provides a novel cyclic carbonates. Cyclic carbonates according to the present invention may be used for the production of polyurethanes. As intermediates in this process urethanes are obtained by a ring opening step. Both the polyurethanes and the urethanes may be used in further processes as they are considered high value products. Crosslinking may also be performed on the polyurethanes and the urethanes. The urethanes and polyurethanes according to the present invention may be used for different applications e.g. foams, seatings, seals, sealants, coatings or adhesives.

The preparation of urethanes and/or polyurethanes may be made without use of phosgene or isocyanate according to the present invention.

### BRIEF DESCRIPTION OF THE DRAWINGS

FIG. 1 discloses general formulas of reactants and products for the formation of cyclic carbonates according to the present invention. Polyol compounds (formula 1a and 1b) forms five and six membered cyclic carbonates (formula 2a and 2b) by reaction with dialkyl or diphenyl carbonate (formula 3). The general formulas may be also their dimer-forms such as ditrimethylopropane and ditrimethylolethane.

R₁, R₂, R₃, R₄, R₅, R₆ may independently be chosen from H, alkyl, hydroxyl, hydroxyalkyl, alkylcarbonyl, carbonylalkyl, alkoxycarbonyl, alkoxycarbonyloxy, carboxyl, allyl, acryl or methacryl group.

FIG. 2 discloses the synthesis of polyurethane and copolymer via TMP-ME cyclic carbonate.

R, R1, R2, R3, R4 may independently be chosen from H, hydroxyalkyl, alkyl, phenyl, hydroxyl, hydroxyalkyl, alkylcarbonyl, carbonylalkyl, alkoxycarbonyl, alkoxycarbonyloxy and carboxyl group.

FIG. 3 discloses FT-IR spectra of the reaction components and products formed during synthesis of polyurethane and copolymer via methacrylated TMP cyclic carbonate. In section (A) TMP-ME, in (B) TMP-ME cyclic carbonate, in (C) TMP-ME mono urethane obtained from reaction with n-hexylamine, in (D) TMP-ME di-urethane obtained from reaction with ethylenediamine, and in (E) polymer from reaction of the material in (D) with ethanedithiol by thermal polymerization.

FIG. 4 discloses a GC chromatogram of TMP-ME cyclic carbonate.

FIG. 5 discloses a ¹H-NMR (A) and ¹³C-NMR (B) of TMP-ME cyclic carbonate.

FIG. 6 discloses a representative GC chromatogram for the reaction of TMP-ME cyclic carbonate with (A) hexylamine (Run 1), and (B) cyclohexylamine (Run 7).

FIG. 7 discloses a representative ¹H and ¹³C-NMR spectrum for the reaction (Run 5). ¹H-NMR of substrate 4b (A), product 6b (B), ¹³C-NMR of substrate 4b (C), and product 6b (D).

FIG. 8 discloses synthesis of polyurethane and copolymer via methacrylated TMP cyclic carbonate.

R, R1, R3 may independently be chosen from H, alkyl, phenyl, hydroxyl, hydroxyalkyl, alkylcarbonyl, carbonylalkyl, alkoxycarbonyl, alkoxycarbonyloxy and carboxyl group.

FIG. 9 discloses FT-IR spectra for synthesis of polyurethane and copolymer via methacrylated TMP cyclic carbonate. (A) TMP, (B) TMP-mMA, (C) TMP-mMA cyclic carbonate, (D) Urethane from reaction of TMP-mMA cyclic carbonate and hexylamine, (E) Diurethane from reaction of TMP-mMA cyclic carbonate and ethylenediamine, and (F) Polymer from reaction of (E) by thermal polymerization.

FIG. 10 discloses GC chromatograms of (A) reaction solution at 24 hr reaction, (B) purified TMP-mMA, and (C) purified TMP-dMA.

FIG. 11 discloses GC chromatograms of TMP-mMA cyclic carbonate.

FIG. 12 discloses a ¹H-NMR (A) and ¹³C-NMR (B) of TMP-mMA cyclic carbonate.

FIG. 13 discloses a representative GC chromatogram for the reaction of TMP-mMA cyclic carbonate with (A) hexylamine (Run 1), and (B) dipropylamine (Run 5).

FIG. 14 discloses representative FT-IR spectra. (A) Substrate 12a and (B) product 14a in Run 1. (C) Substrate 11a and (D) product 13a in Run 5.

FIG. 15 discloses the synthesis of polyurethane and copolymer via TMP cyclic carbonate.

R, R1, R2, R3 may independently be chosen from H, alkyl, phenyl, hydroxyl, hydroxyalkyl, alkylcarbonyl, carbonylalkyl, alkoxycarbonyl, alkoxycarbonyloxy and carboxyl group.

FIG. 16 discloses FT-IR spectra of the reaction components and products formed during synthesis of polyurethane and copolymer from TMP cyclic carbonate. (A) TMP-CC, (B) TMP diurethanes ring-opened by diamines, polyurethanes from diurethanes.

FIG. 17 discloses a general polymerization process from six-membered bicyclic carbonates with diamines.

R=oxygen (ether), alkyl (0-10 carbons), ketone, ester. R₁, R₂ may independently be H, alkyl, hydroxyl, hydroxyalkyl, alkylcarbonyl, carbonylalkyl, alkoxycarbonyl, alkoxycarbonyloxy and carboxyl group, independently. R3=alkyl (1-20 carbons), cycloalkyl, alkylphenyl (e.g. xylylenediamine), isophorone, polyamines (e.g. Jeffamine ED-600) and derivatives thereof.

FIG. 18 discloses representative FT-IR spectra of the reaction components and polyurethane products formed during polymerization of diTMP dicyclic carbonate (diTMPdiCC) with xylylenediamine in dichloromethane at RT. (A) diTMPdiCC, (B) XDA, (C) diTMPdiCC and XDA (D) homogenized solution of diTMPdiCC and XDA in dichloromethane (reaction time 0 minute), (E) homogenized solution of diTMPdiCC and XDA in dichloromethane (reaction time 1 minute), and (F) homogenized solution of diTMPdiCC and XDA in dichloromethane (reaction time 5 minute).

FIG. 19 discloses representative FT-IR spectra of the reaction components and polyurethane products formed on the glass surface during coating application by polymerization of diTMP dicyclic carbonate with xylylenediamine at 110° C. (A) diTMPdiCC and XDA (B) Run 1 after drying for 2 days (Table 5): A shifted strong peak in 1690 cm⁻¹ indicates an amide bond of urethane group and a new strong peak at 3000-3500 cm⁻¹ appeared for —OH group resulted from ring opening of cyclic carbonate.

FIG. 20 discloses representative FT-IR spectra of the reaction components and polyurethane products formed on the glass surface during coating application by polymerization of diTMP dicyclic carbonate with xylylenediamine in dichloromethane at 110° C. (A) diTMPdiCC and XDA (B) Run 5 at drying 2 days (Table 8): A shifted strong peak in 1690 cm⁻¹ indicates an amide bond of urethane group and a new strong peak at 3000-3500 cm⁻¹ appeared for —OH group resulted from ring opening of cyclic carbonate.

FIG. 21 discloses representative FT-IR spectra of the reaction components and polyurethane products formed on the glass surface during coating application by polymerization of diTMP dicyclic carbonate with xylylenediamine in dichloromethane at 60° C. (A) diTMPdiCC and XDA (B) Run 5 at drying 2 days (Table 9): A shifted strong peak in 1690 cm⁻¹ indicates an amide bond of urethane group and a new strong peak at 3000-3500 cm⁻¹ appeared for —OH group resulted from ring opening of cyclic carbonate.

FIG. 22 discloses representative FT-IR spectra of the reaction components and polyurethane products formed on the glass surface during coating application by polymerization of diTMP dicyclic carbonate with xylylenediamine in dichloromethane at RT. (A) diTMPdiCC and XDA (B) Run 5 after drying for 2 days (Table 10): A shifted strong peak in 1690 cm⁻¹ indicates an amide bond of urethane group and a new strong peak at 3000-3500 cm⁻¹ appeared for —OH group resulting from ring opening of cyclic carbonate.

### DETAILED DESCRIPTION

A more complete appreciation of the invention, and many of the attendant advantages thereof, will be readily apparent and better understood by reference to the following detailed description and figures.

The wordings “bicyclic” and “dicyclic” carbonates are used interchangably herein.

One object of the present invention is to provide a method of producing a functionalized cyclic carbonate comprising the steps of:

a) providing a polyol having at least one functional group chosen from the group of hydroxyl, alkylhydroxyl, allyl, allylether, acryl, and methacryl;
b) admixing a dialkyl carbonate or a biphenyl carbonate and said functionalizes polyol of a) forming a mixture;
c) heating the mixture of b) to obtain a cyclic functionalized carbonate.

According to one embodiment the polyol of a) comprises at least three carbon atoms.

According to one embodiment the polyol preferably contains at least two hydroxyl groups connected to at least two of the mentioned three carbon atoms.

According to one embodiment the method comprises the steps of:

a) providing a polyol having a formula selected from

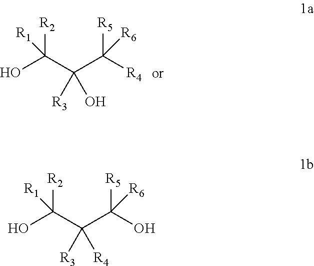

having at least one functional group chosen from the group of hydroxyl, alkylhydroxyl, allyl, allylether, acryl, and methacryl;
b) admixing a dialkyl carbonate or a biphenyl carbonate and said functionalized polyol of a) forming a mixture;
c) heating the mixture of b) to obtain a cyclic functionalized carbonate having a formula selected from

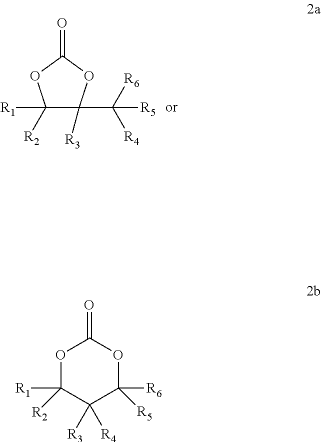

or their corresponding dimers.

According to one embodiment the the polyol of a) is obtained by providing and admixing a polyol and a compound having at least one functional group chosen from the group of hydroxyl, alkylhydroxyl, allyl, allylether, acryl, and methacryl.

According to one embodiment the polyol used to form the cyclic carbonated may be chosen from polyols having 2 to 8 hydroxy groups, preferably polyols having 2 to 6 hydroxy groups, more preferably polyols having 2 to 4 hydroxy groups.

According to one embodiment the polyol and/or the cyclic carbonate, respectively may contain substituents chosen from H, alkyl, aryl, hydroxyl, hydroxyalkyl, alkylcarbonyl, carbonylalkyl, alkoxycarbonyl, alkoxycarbonyloxy and carboxyl, preferably all substituents are independently chosen from H, alkyl, hydroxyl, hydroxyalkyl, alkylcarbonyl, carbonylalkyl, alkoxycarbonyl, alkoxycarbonyloxy and carboxyl.

According to one embodiment the said heating is performed at a temperature of at least 80° C., preferably at least 90° C., preferably at least 100° C., preferably at least 120° C., preferably at least 140° C.

According to one embodiment the the obtained functionalized cyclic carbonates were collected via a separation process.

According to one embodiment the the separation process is chosen from at least one of decantation, filtration, centrifugation, evaporation, preferably a combination of filtration and evaporation.

According to one embodiment the separation process is followed by a purification step.

According to one embodiment the purification step is precipitation, recrystallization and/or chromatography, preferably column silica flash chromatography.

Another object of the present invention is to provide a method of producing a functionalized monourethane and/or diurethane comprising the steps of:

i) providing a functionalized cyclic carbonate;
ii) providing at least one compound selected from the group alkylamine, aromatic amine and diamine;
iii) forming a mixture of said carbonate of i) and said at least one compound of ii);
iv) allowing reaction of the mixture of iii) by ring opening;
v) obtaining a functionalized monourethane and/or diurethane.

According to one embodiment the reaction by ring opening of step iv) is performed in the absence or presence of a catalyst.

According to one embodiment the reaction by ring opening of step iv) is performed in the absence or presence of an organic solvent.

According to one embodiment the organic solvent may be chosen from dimethylformamide, dimethylsulfoxide, pyridine and acetonitrile.

According to one embodiment the alkylamine may be chosen from hexylamine, cyclohexylamine and dipropylamine.

According to one embodiment the diamine may be chosen from alkyldiamines, preferably 1,6-hexamethylenediamine, 1,2-diethylenediamine and isophorone diamine.

According to one embodiment the reaction by ring opening of step iv) is performed at a temperature of at least 20° C., preferably at least 50° C., preferably at least 100° C., preferably at least 120° C., preferably at least 140° C.; or at a temperature of at most 0° C., preferably at most −10° C.

Another object of the present invention is to provide a method of producing a polyurethane comprising the steps of:
A) providing a functionalized urethane and/or diurethane;
B) reacting the a functionalized urethane and/or diurethane of A) in at least one step by UV and/or thermal reaction and/or isocyanate; and
C) obtaining a polyurethane.

According to one embodiment the reaction of step B) additionally involves a thiol compound.

According to one embodiment the thiol compound is chosen from polythiols.

According to one embodiment the polythiol compound is chosen from
dithiols, preferably 1,2-ethylendithiol,
trithiols, preferably trimethylolpropane tris(3-mercaptopropionate),
tetrathiols, preferably pentaerythritol tetrakis (3-mercaptopropionate).

According to one embodiment an initiator is used in the reaction of step D).

According to one embodiment the initiator is selected from the group azo compounds of azobisisobutyronitrile (AIBN) and 1,1′-azobis(cyclohexanecarbonitrile) (ABCN), and organic peroxides of di-ter-butyl peroxide and benzoyl peroxide.

According to one embodiment the reaction of step D) is performed in the absence or presence of an organic solvent.

According to one embodiment the organic solvent is chosen from dimethylformamide, dimethylsulfoxide, chloroform, pyridine and acetonitrile.

According to one embodiment when the reaction of step D) is performed using thermal energy the temperature is at least 20° C., preferably at least 90°, preferably at least 100° C., preferably at least 120°, or preferably at least 140° C.

Another object of the present invention is to provide a method of producing a polyurethane comprising the steps of:

m) providing a bicyclic carbonate having the formula

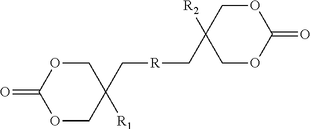

wherein R is chosen from a bond, oxygen, alkyl having 1-10 carbons, ketone, and ester; R₁ and R₂ may independently be chosen from H, alkyl, hydroxyl, hydroxyalkyl, alkylcarbonyl, carbonylalkyl, alkoxycarbonyl, alkoxycarbonyloxy and carboxyl group; and R3 is chosen from alkyl having 1-20 carbons, cycloalkyl, alkylphenyl, isophorone, polyamines, and derivatives thereof;
n) providing a diamine;
o) forming a mixture of said bicyclic carbonate of m) and said diamine of n);
p) allowing reaction of the mixture of o);
q) obtaining a polyurethane having the formula

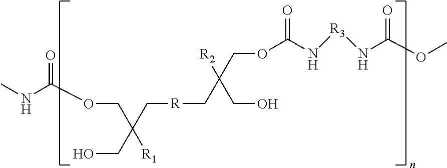

wherein R is chosen from a bond, oxygen, alkyl having 1-10 carbons, ketone, ester,
R₁, R₂ independently are chosen from H, alkyl, hydroxyl, hydroxyalkyl, alkylcarbonyl, carbonylalkyl, alkoxycarbonyl, alkoxycarbonyloxy and carboxyl group,
R3 is chosen from alkyl having 1-20 carbons, cycloalkyl, alkylphenyl, isophorone, polyamines and derivatives thereof.

According to one embodiment the reaction in p) is done in presence or absence of a solvent.

According to one embodiment the solvent comprises alcohols, preferably methanol, ethanol and propanol; (cyclic) ethers, preferably diethyl ether and tetrahydrofuran (THF); ketones, preferably acetone, ethylmethylketone; toluene; acetonitrile; halogenated alkane, preferably dichloromethane or chloroform; dimethylformamide; pyridine; or mixtures thereof.

According to one embodiment said solvent consists of at least one of the solvents mentioned above.

According to one embodiment the reaction in p) is done in presence or absence of a catalyst.

According to one embodiment the diamine is chosen from the group alkyldiamine, preferably 1,6-hexamethylenediamine, 1,2-diethylenediamine and isophorone diamine; or phenylalkyldiamine, preferably xylylenediamine.

Another object of the present invention is to provide a method of producing crosslinked polyurethanes or copolymers comprising the steps of:

I) providing a functionalized urethane and/or diurethane or a polyurethane;
II) reacting the functionalized urethane and/or diurethane or polyurethane of I) by UV and/or thermal reaction or isocyanate;
III) obtaining a crosslinked polyurethane or copolymer product.

According to one embodiment the reaction in step II) include a thiol compound.

According to one embodiment the isocyanate compound is a polyisocyanate, preferably chosen from diisocyanate, preferably chosen from 1,6-hexamethylenediisocyanate, 1,2-diethylenediisocyanate, isophorone diisocyanate, and toluene-2,4-diisocyanate.

Another object of the present invention is to provide a cyclic carbonate comprising functional groups selected from the group hydroxyl, alkylhydroxyl, allyl, allylether, acryl, methacryl.

According to one embodiment the cyclic carbonate all substituents are independently chosen from hydroxyl, alkylhydroxyl, allyl, allylether, acryl, and methacryl.

According to one embodiment the cyclic carbonates are mono or multicyclic carbonates, preferably mono, bi, tri or tetracyclic carbonates, more preferably mono, bi or tricyclic carbonates, more preferably mono or bicyclic carbonates, more preferably bicyclic carboantes.

According to one embodiment the cyclic carbonates have at least one five-membered or six-membered ring from a polyol, preferably at least one six-membered ring from a polyol.

Another object of the present invention is to provide a cyclic carbonate obtained by the method mentioned above.

According to one embodiment the cyclic carbonate is 5-membered or 6-membered cyclic carbonate, preferably 6-membered cyclic carbonate.

According to one embodiment the cyclic carbonate is monocyclic or multicyclic, preferably comprising 1 to 4 cyclic moieties, preferably comprising 1 to 3 cyclic moieties.

According to one embodiment the cyclic carbonate has a formula of

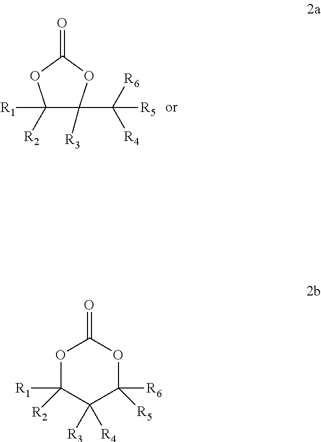

or their corresponding dimers
wherein R₁, R₂, R₃, R₄, R₅, R₆ independently are chosen from H, alkyl, hydroxyl, hydroxyalkyl, alkylcarbonyl, carbonylalkyl, alkoxycarbonyl, alkoxycarbonyloxy, carboxyl, allyl, acryl and methacryl, and
at least one of R₁, R₂, R₃, R₄, R₅ and R₆ is chosen from hydroxyl, hydroxyalkyl, allyl, allylether, acryl or methacryl group or

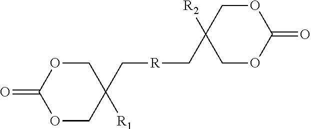

wherein R is chosen from a bond, oxygen, alkyl having 1-10 carbons, ketone, and ester; R₁ and R₂ may independently be chosen from H, alkyl, hydroxyl, hydroxyalkyl, alkylcarbonyl, carbonylalkyl, alkoxycarbonyl, alkoxycarbonyloxy and carboxyl group; and R3 is chosen from alkyl (1-20 carbons), cycloalkyl, alkylphenyl, isophorone, polyamines and derivatives thereof.

According to one embodiment the cyclic carbonate may be functionalized by groups selected from allyl, allylether, acryl and methacryl.

Another object of the present invention is to provide a functionalized monourethane and/or diurethane having a formula chosen from

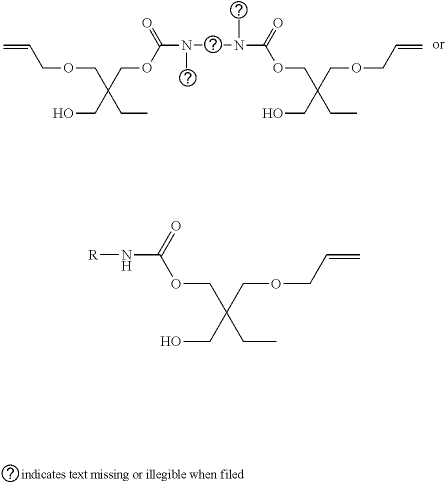

wherein R, R1, R2, R3, and R4 independently are chosen from H, hydroxyalkyl, alkyl, phenyl, hydroxyl, hydroxyalkyl, alkylcarbonyl, carbonylalkyl, alkoxycarbonyl, alkoxycarbonyloxy and carboxyl;

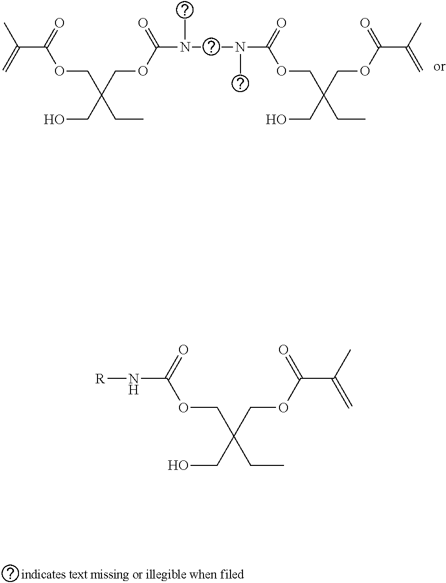

wherein R, R1, and R3 independently are chosen from H, alkyl, phenyl, hydroxyl, hydroxyalkyl, alkylcarbonyl, carbonylalkyl, alkoxycarbonyl, alkoxycarbonyloxy and carboxyl;
or

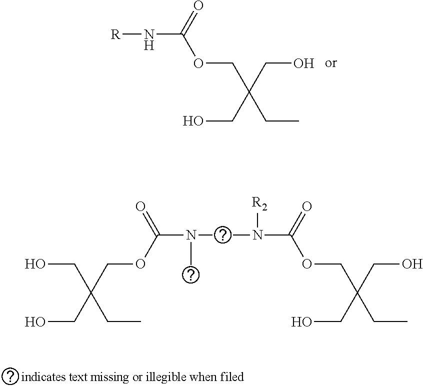

wherein R, R1, R2, and R3 independently are chosen from H, alkyl, phenyl, hydroxyl, hydroxyalkyl, alkylcarbonyl, carbonylalkyl, alkoxycarbonyl, alkoxycarbonyloxy and carboxyl group.

Another object of the present invention is to provide a functionalized monourethane and/or diurethane obtained by the method mentioned above.

Another object of the present invention is to provide a polyurethane having a formula chosen from

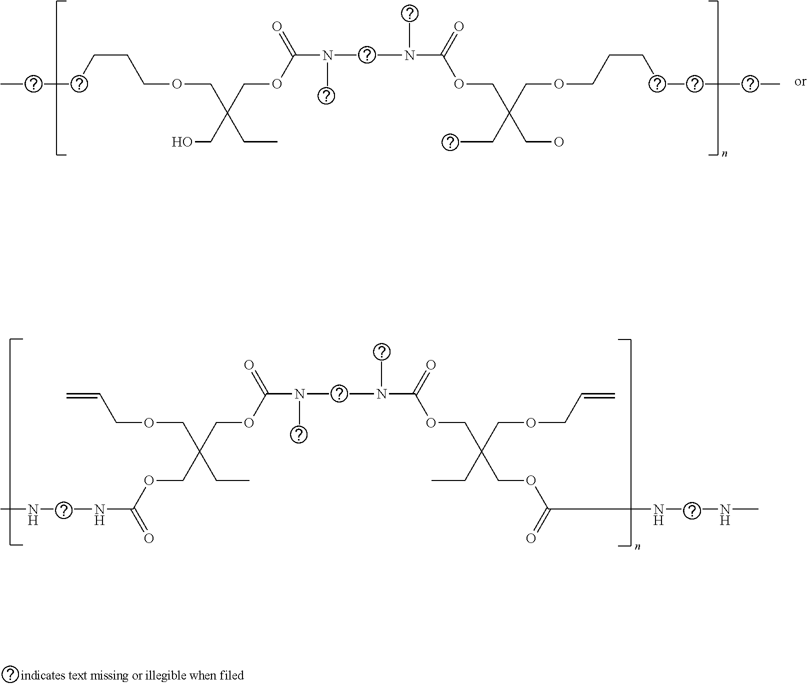

wherein R, R1, R2, R3, and R4 independently are chosen from H, hydroxyalkyl, alkyl, phenyl, hydroxyl, hydroxyalkyl, alkylcarbonyl, carbonylalkyl, alkoxycarbonyl, alkoxycarbonyloxy and carboxyl;

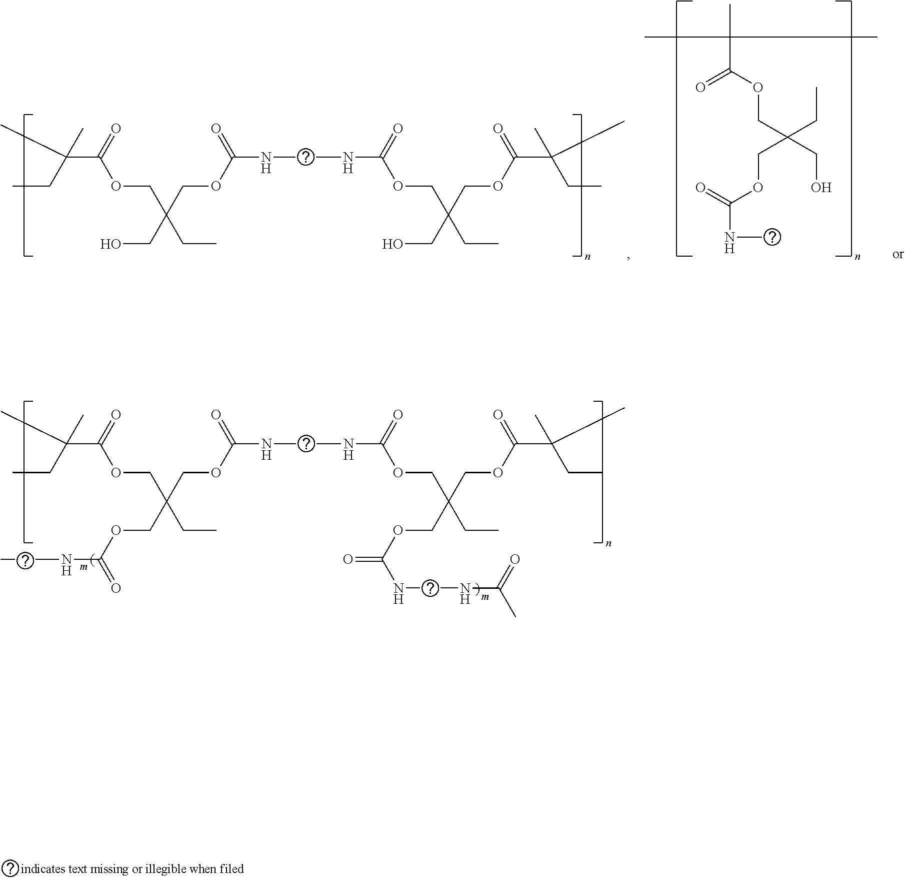

wherein R, R1, and R3 independently are chosen from H, alkyl, phenyl, hydroxyl, hydroxyalkyl, alkylcarbonyl, carbonylalkyl, alkoxycarbonyl, alkoxycarbonyloxy and carboxyl;

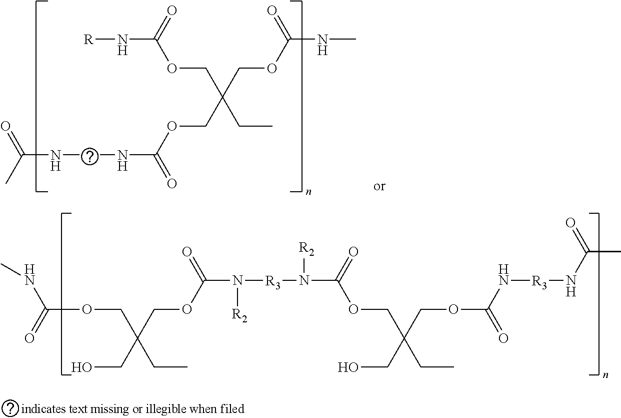

wherein R, R1, R2, and R3 independently are chosen from H, alkyl, phenyl, hydroxyl, hydroxyalkyl, alkylcarbonyl, carbonylalkyl, alkoxycarbonyl, alkoxycarbonyloxy and carboxyl;
or

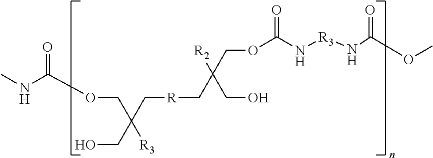

wherein R is chosen from a bond, oxygen, alkyl having 1-10 carbons, ketone, ester,
R₁, R₂ independently are chosen from H, alkyl, hydroxyl, hydroxyalkyl, alkylcarbonyl, carbonylalkyl, alkoxycarbonyl, alkoxycarbonyloxy and carboxyl group,
R3 is chosen from alkyl having 1-20 carbons, cycloalkyl, alkylphenyl, isophorone, polyamines and derivatives thereof.

Another object of the present invention is to provide a polyurethane obtained by the method mentioned above.

Another object of the present invention is to use a functionalized urethane and/or diurethane or a polymerized functionalized monourethane and/or diurethane for the production of foams, seatings, seals, sealants, coatings or adhesives.

According to one embodiment materials mentioned are used for the production of insulation foams, packaging foames, structural foam, high resiliency foam, footwear soles, simulated wood, integral skin foam for vehicle interiors, facia and other exterior parts of a vehicle, durable elastomeric wheels and tires, synthetic fibers, print rollers, cast elastomers, reaction injection molded plastic, material enclosing electronic components or implants and devices of medical use. According to the present invention five-membered or six-membered cyclic carbonates may be used, preferably six-membered cyclic carbonates. Six-membered cyclic carbonates are preferred to the five-membered ones because of them being less thermodynamically stable than its ring-opened polymer and thus retaining CO₂, during the polymerization process. The reactivities of six membered compared to five membered cyclic carbonates with functional groups are considerably higher. The reaction rate of the six-membered cyclic carbonate may at a temperature of about 30-70° C. be about 30 to 60 times higher than those of the five membered ones.

Cyclic carbonates can be further functionalized with active groups such as allyl and methacryl groups for UV or thermal reaction and polymerization with initiator. Allyl group reacts with thiol group by the thiolene reaction mechanism by UV or thermal reaction. Acrylate and methacrylate are common monomers in polymer plastics, forming the corresponding polymers because the α,β-unsaturated double bonds are very reactive. Hydroxyl urethanes and polyhydroxurethanes obtained by ROP of cyclic carbonates can react with isocyanates. Cyclic carbonates, functionalized cyclic carbonates and ring opened hydroxyurethanes could be useful building blocks in chemistry and polymer industry.

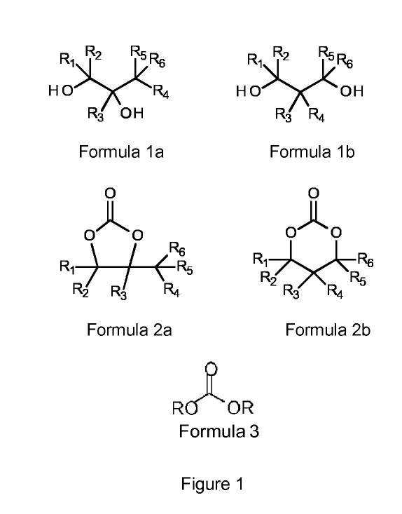

In the present invention, cyclic carbonates are preferably monocyclic and/or polycyclic carbonates having a five-membered or six-membered ring from a polyols. According to one embodiment the cyclic carbonates are chosen from monocyclic and/or bicyclic carbonates. The polyols may preferably be diols, triols or tetraols. Polyols could be a 2,2-dialkyl-1,3-propanediol, 2-alkyl-2-hydroxyalkyl-1,3-propanediol, 2,2-hydroxylalkyl-1,3-propanediol, which can be exemplified by neo-pentyl glycol, 2-butyl-2-ethyl-1,3-propaneiol, trimethylolpropane, trimethylolethane, pentaerythritol, and their dimer forms such as di-trimethylolpropane. Further suitable polyols include glycerol, sorbitol, mannitol, and derivatives thereof (see FIG. 1).

Cyclic carbonates could be mono and multi-functionalized with allyl, allylether, acryl and methacryl groups. (R₁, R₂, R₃, R₄, R₅, R₆ may independently be chosen from hydroxyl, alkylhydroxyl, allyl, allylether, acryl, and methacryl, see FIG. 1).

Cyclic carbonate functionalized with hydroxyl, allyl, acryl and methacryl groups are unique monomers and/or linkers for polymerization, can be used for producing polyurethanes, polycarbonates, and copolymers.

In the functionalized cyclic carbonate, cyclic carbonate group reacts with alkyl and aromatic amine, and diamine compounds by ring opening reaction to produce urethane and diurethane bonds, respectively. The resulting functionalized urethane and diurethane products are unique monomers and/or linkers for polymerization, and can be reacted or polymerized using allyl, acryl and methacryl groups with an initiator by UV and thermal reaction.

The present invention provides a facile, green and cost effective production method of polyurethanes and copolymers from functionalized cyclic carbonates.

According to the present invention five-membered or six-membered cyclic carbonates are used, preferably six-membered cyclic carbonates.

For use in ROP process, however, six-membered cyclic carbonates are preferred to the five-membered one because of being less thermodynamically stable than its ring-opened polymer and thus retaining CO2, during the polymerization process.

The polyurethane materials prepared in accordance with the present invention may be used as foams, seatings, seals and sealants, high performance coatings and adhesives.

Foams may be insulation foams, packaging foames, structural foam, high resiliency foam for bedding and upholstery. Further uses of said polyurethane materials may be footwear soles (outsoles and midsoles), simulated wood, integral skin foam for vehicle interiors, facia and other exterior parts of a vehicle, durable elastomeric wheels and tires, synthetic fibers, print rollers, cast elastomers, reaction injection molded plastic, and material enclosing electronic components. Said polyurethane materials may be used for biomedical purposes e.g. in implants or other devices of medical use.

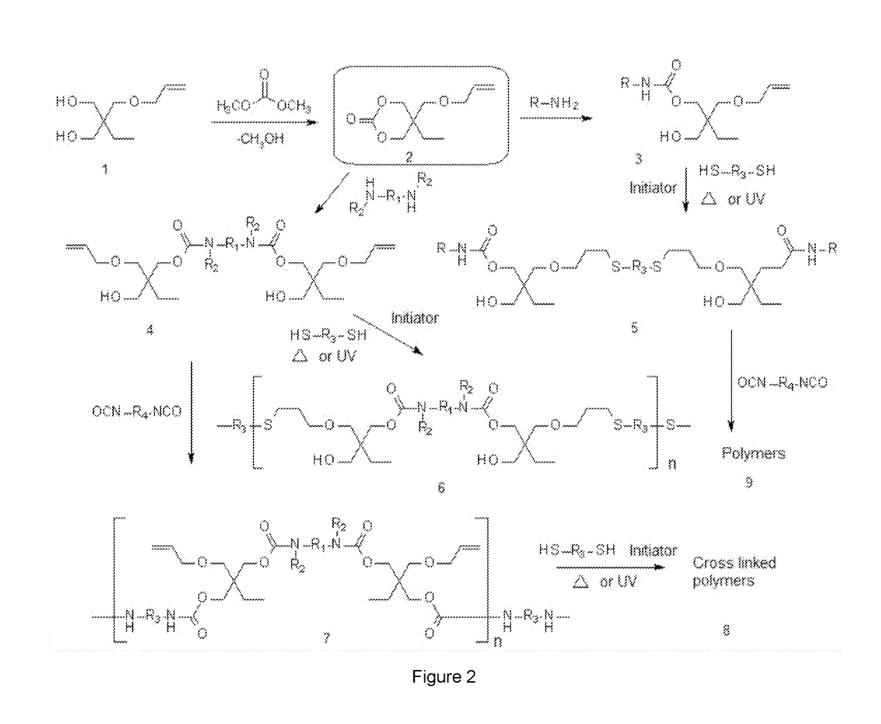

One object of the present invention is to provide allylated polyol cyclic carbonate, manufacturing polyurethanes and copolymers thereof, and crosslinking the building blocks with isocyanate compounds. Below reference is made to FIG. 2.

1.1 Synthesis of Monoallylated TMP Cyclic Carbonate

Cyclic carbonates (2) may be prepared from trimethylolpropane monoallylether (TMPME) and dimethylcarbonate (DMC). TMP may be reacted with DMC in a reaction vessel. The ratio of TMPE to DMC may be 1 to 20. The rectants may be admixed with molecular sieves. The ratio of reactants to molecular sieves may be 1 to 20. The reaction between TMP and DMC, optionally including molecular sieves, may be performed at a temperature of at least 80° C., preferably at least 90° C., preferably at least 100° C., preferably at least 120° C., preferably at least 140° C. The result of the reaction between TMPE and DMC is a functionalized cyclic carbonate: monoallylated TMP cyclic carbonate. The obtained functionalized cyclic carbonate may be collected via a separation process. The separation process may be chosen from at least one of decantation, filtration, centrifugation, evaporation, preferably filtration and/or evaporation.

1.2 Ring Opening Reaction with Amine and Diamine Compounds

Monoallylated TMP cyclic carbonate may be reacted with amine or diamine compounds in the absence or presence of a catalyst, resulting in a ring opening reaction. The temperature during a reaction between the cyclic carbonate and amine or diamine compounds may be at most 0° C., such as at most −10° C. The temperature during a reaction between the cyclic carbonates and amine or diamine compounds may be at least 20° C., such as at least 100° C., at least 120° C., or at least 140° C. The reaction by ring opening may be performed in the absence or presence of an organic solvent. The organic solvent may be chosen from dimethylformamide (DMF), dimethylsulfoxide (DMSO), pyridine and acetonitrile. The amine may be an alkylamine and may be chosen from hexylamine, cyclohexylamine and dipropylamine. The diamine may be an alkyldiamine, and may be chosen from 1,6-hexamethylenediamine, 1,2-diethylenediamine and isophorone diamine. In the ring opening reaction a mono-urethane (3) from the amine reaction and a di-urethane (4) from the diamine reaction may be formed.

1.3 Polymerization with the Ene Functional Group in Ring-Opened Mono- and Di-Urethane TMPME

The obtained ring opened mono- and diurethane TMPME may be polymerized via an ene functional group. The ring opened mono- (3) and diurethane TMPME (4) may be reacted with thiol compounds using UV or thermal energy. The thiol compounds may be chosen from dithiols, such as 1,2-ethylendithiol; or trithiols, such as trimethylolpropane tris(3-mercaptopropionate); tetrathiols, such as pentaerythritol tetrakis (3-mercaptopropionate); and polythiols. An initiator may be used in the reaction and polymerization process, which may be selected from the group azo compounds of azobisisobutyronitrile (AIBN) and 1,1′-azobis(cyclohexanecarbonitrile) (ABCN), and organic peroxides of di-ter-butyl peroxide and benzoyl peroxide. The mentioned reaction and polymerization may be carried out in solvent. The organic solvent may be chosen from DMF, DMSO, pyridine, chloroform and acetonitrile. If the reaction and polymerization is performed using thermal energy the temperature may be at least 20° C., such as at least 90°, at least 100° C., at least 120°, or at least 140° C.

1.4 Polymerization with the Hydroxyl Group in Ring-Opened Mono- and Di-Urethane TMPME

The obtained ring opened mono- and diurethane TMPME may be polymerized via an hydroxyl functional group. The di-urethane (4, 5) may be polymerized with isocyanate compounds. Diurethane (4,5) may be reacted with di- and/or polyisocyanate compounds in the absence or presence of catalyst. The temperature during such a reaction may be at least 20° C., preferably at least 50° C., preferably at least 100° C., preferably at least 120° C., preferably at least 140° C. Alternatively, the temperature during such a reaction may be at most 0° C., preferably at most −10° C. The polymerization may be performed in the absence or presence of an organic solvent. The organic solvent may be chosen from DMF, DMSO, pyridine, and acetonitrile.

Isocyanate compounds that may be used for the polymerisation process may be 1,6-hexamethylenediisocyanate, 1,2-diethylenediisocyanate, isophorone diisocyanate, and toluene-2,4-diisocyanate. By the mentioned reaction, polyurethanes (7 or 9) may be formed. Polyurethanes (7) can further be crosslinked by thiol compounds.

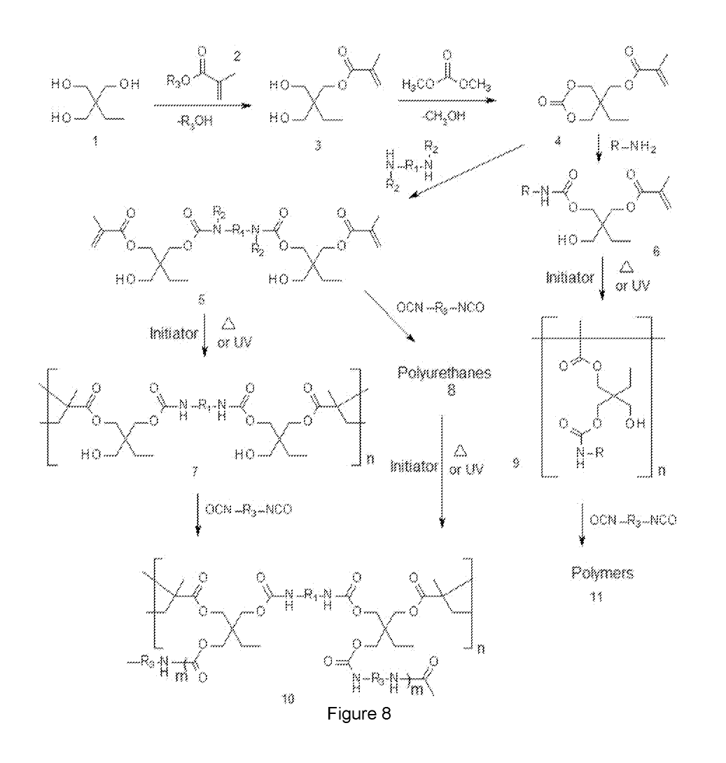

One object of the present invention is to provide acrylated or methacrylated polyol cyclic carbonate, manufacturing polyurethanes and copolymers thereof, and crosslinking the building blocks with isocyanate compounds. Below reference is made to FIG. 8.

### 2.1 Synthesis of Methacrylated TMP

Methacrylated TMP (TMP-mMA, 3) is not commercially available. TMP may be dissolved in methacrylate ester. The ratio of TMP to methacrylate ester may be 1 to 20. The reaction may be performed in a reaction vessel with stirring and heating, e.g. magnetic stirring in an oil bath at 60° C. The reaction may be initiated by addition of an enzyme, e.g. Novozym 435 at 10% (w/w) of TMP. The reactants may be admixed with molecular sieves. The ratio of reactants to molecular sieves may be 1 to 10. The reaction between TMP and methacrylate ester, optionally including molecular sieves, may be performed at a temperature of at least 30° C., such as at least 50° C. or at least 70° C. The methacrylate may be acid and/or esters, e.g. chosen from methyl, ethyl and vinyl ester. Aliquots may be withdrawn at different time intervals for analysis of the reaction components. After completion of the reaction, the products may be collected via a separation process. The separation process may be chosen from at least one of decantation, filtration, centrifugation, evaporation, preferably filtration and/or evaporation. The separation process may thus be filtration of residual solid and/or evaporation of excess methyl methacrylate. The resulting TMP-mMA may be purified. Purification may be made using column silica flash chromatography. Ethyl acetate and a mixture of ethyl acetate and methanol (1:1) may be used as eluent

The preparation of methacrylated TMP may also be achieved by a chemical process with protection of a diol. The diol-protected TMP may then be transesterified using a methacrylic acid, esters such as methyl, ethyl and vinyl ester, and followed by deprotection.

### 2.2 Synthesis of Methacrylated TMP Cyclic Carbonate

The purified TMP-mMA may be converted to the corresponding six-membered cyclic carbonate. The cyclic carbonates may be prepared by reacting TMP-mMA and dimethylcarbonate (DMC). The ratio of TMP-mMA to DMC may be 1 to 20. The reactant solution may contain molecular sieves. The ratio of reactant solution to molecular sieves may be 1 to 20. The reactant solution, optionally containing molecular sieves, may be heated. The temperature during reaction may be at least 80° C., such as at least 90° C., at least 100° C., at least 120° C., or at least 140° C. After completion of the reaction, TMP-mMA cyclic carbonate (4) may be collected via a separation process. The separation process may be chosen from at least one of decantation, filtration, centrifugation, evaporation, preferably filtration and/or evaporation.

2.3 Ring Opening Reaction with Amine and Diamine Compounds

TMP-mMA cyclic carbonate (4) may be reacted with amine or diamine compounds in the absence or presence of catalyst. The temperature during such a reaction may be at least 20° C., preferably at least 50° C., preferably at least 100° C., preferably at least 120° C., preferably at least 140° C. Alternatively, the temperature during such a reaction may at most 0° C., preferably at most −10° C. The reaction may be performed in the absence or presence of an organic solvent. The organic solvent may be chosen from DMF, DMSO, pyridine, and acetonitrile. Amine compounds may be chosen from alkylamines, e.g. chosen from hexylamine, cyclohexylamine and dipropylamine. Diamine compounds may be chosen from alkyldiamines, e.g. chosen from 1,6-hexamethylenediamine, 1,2-diethylenediamine xylylenediamine and isophorone diamine. By the reaction, mono-urethane (6) from amine reaction and diurethane (5) from diamine reaction may be formed.

### 2.4 Polymerization at the Methacrylate Functional Group in Ring-Opened Mono- and Diurethanes.

The obtained ring opened mono- and diurethanes of TMP-mMA cyclic carbonate may be polymerized via an methacrylate functional group. Mono-urethane (6) and di-urethane (5) may be polymerized by UV or thermal reaction. The UV or thermal reaction may be initiated by an initiator. The initiator may be selected from azo compounds of azobisisobutyronitrile (AIBN) and 1,1′-azobis(cyclohexanecarbonitrile) (ABCN), and organic peroxides of di-ter-butyl peroxide and benzoyl peroxide. The reaction and polymerization may be performed in the absence or presence of an organic solvent. The organic solvent may be DMF, DMSO, pyridine, chloroform and acetonitrile. If the reaction and polymerization is performed using thermal energy the temperature may be at least 20° C., such as at least 90° C., at least 100° C., at least 120° C., or at least 140° C. Any typical polymerization method may be used in the polymerization of methacrylate by UV and/or thermal reaction in the absence or presence of an initiator and/or catalyst. By polymerization, copolymers (7) from di-urethanes (5) and copolymers (9) from mono-urethanes (6) may be formed.

Polymers (7,9) may be further crosslinked with the use of isocyanates. Hydroxyl groups in the obtained polymers (7,9) may be further reacted with di- and polyisocyanate compounds in the absence or presence of catalyst. The mentioned crosslinking may be performed at a temperature of at least 20° C., preferably at least 50° C., preferably at least 100° C., preferably at least 120° C., preferably at least 140° C. Alternatively, the temperature during such a reaction may be at most 0° C., preferably at most −10° C. The crosslinking reaction may be performed in the absence or presence of an organic solvent. The organic solvent may be DMF, DMSO, pyridine, and acetonitrile. Isocyanate compounds used in the crosslinking reaction may be chosen from 1,6-hexamethylenediisocyanate, 1,2-diethylenediisocyanate, isophorone diisocyanate, and toluene-2,4-diisocyanate. By the crosslinking reaction, copolymers (10 or 11) may be formed.

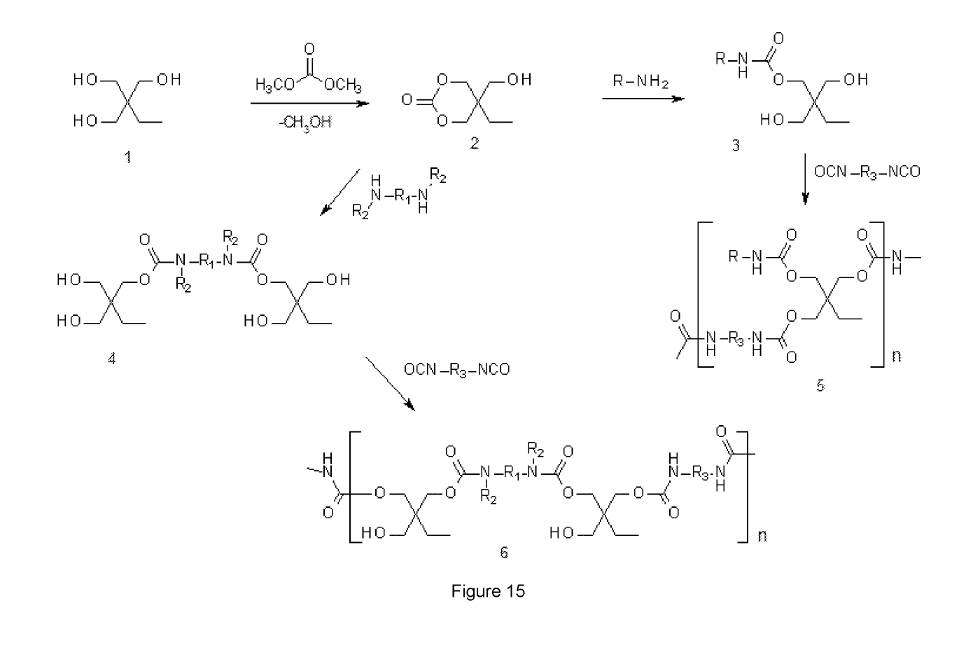

One object of the present invention is to provide hydroxyl cyclic carbonate, manufacturing polyurethanes and copolymers thereof, and crosslinking the building blocks with isocyanate compounds. Below reference is made to FIG. 15.

### 3.1 Synthesis of TMP Cyclic Carbonate

Cyclic carbonates may be prepared from TMP and dimethylcarbonate (DMC). TMP is commercially available. TMP may be reacted with DMC at ratio of 1 to 20. The reactant solution comprising TMP and DMC may be admixed with molecular sieves. The ratio of reactant solution to molecular sieves may be 1 to 20. The reactant solution, optionally comprising molecular sieves, may be heated. Said heating may be performed at a temperature of at least 80° C., such as at least 90° C., at least 100° C., at least 120° C. or at least 140° C. After completion of the reaction, crude TMP cyclic carbonate may be collected via a separation process. The separation process may be chosen from at least one of decantation, filtration, centrifugation, evaporation, preferably filtration and/or evaporation. The obtained TMP cyclic carbonate may be purified. Purification may be performed using silica fresh chromatography, preferably in a temperature range of at least −20° C., such as at least 0° C., or at least 20° C. The chromatography temperature may preferably be in range of −10° C. to 30° C.

According to one embodiment crude TMP cyclic carbonate dissolved in ethylaacetate may be loaded into a column packed with silica gel (equilibrated with ethylacetate). Ethylacetate was used as a mobile phase. The flow rate was accelerated by blowing nitrogen. Eluted solution was fractionated, and analysed using GC. Then the column was washed using methanol, and reused after being equilibrated with EA.

As a specific example, 5 g crude TMP cyclic carbonate dissolved in 50 mL ethylacetate was loaded into 10 cm (ID)×14 cm (L) column packed with silica gel (Merck), which was equilibrated with ethylacetate (EA). Ethylacetate was used as a mobile phase. The flow rate was accelerated by blowing nitrogen. Eluted solution was fractionated, and analysed using GC. Then the column was washed using methanol, and reused after equilibrated with EA.

3.2 Ring Opening Reaction of TMP Cyclic Carbonate with Amine and Diamine Compounds

TMP cyclic carbonates may be reacted with amine or diamine compounds in the absence or presence of catalyst. The ring opening reaction may be performed at a temperature of at least 20° C., preferably at least 50° C., preferably at least 100° C., preferably at least 120° C., preferably at least 140° C. Alternatively, the temperature during such a reaction may be at most 0° C., preferably at most −10° C. The ring-opening reaction may be performed in the absence or presence of an organic solvent. The organic solvent may be DMF, DMSO, pyridine, and acetonitrile. Amine compounds may be chosen from alkylamines, e.g. chosen from hexylamine, cyclohexylamine and dipropylamine. Diamine compounds may be chosen from alkyldiamines, e.g. chosen from 1,6-hexamethylenediamine, 1,2-diethylenediamine, isophorone diamine and N,N′-di-n-propylethylenediamine. By the ring-opening reaction, mono-urethane (3) from amine reaction and diurethane (4) from diamine reaction may be formed.

3.3 Polymerization of Urethane Compounds (4, 6) with Isocyanate

Diisocyanate compounds may be used in reaction and polymerization of mono-urethane and di-urethane. Mono-urethane (3) and diurethane (4) may be reacted with di- and polyisocyanate compounds in the absence or presence of catalyst. The reaction may be performed at a temperature of at least 20° C., preferably at least 50° C., preferably at least 100° C., preferably at least 120° C., preferably at least 140° C. Alternatively, the temperature during such a reaction may be at most 0° C., preferably at most −10° C. The reaction may be performed in the absence or presence of an organic solvent. The organic solvent may be DMF, DMSO, pyridine, and acetonitrile. Isocyanate compounds used in the reaction may be chosen from 1,6-hexamethylenediisocyanate, 1,2-diethylenediisocyanate, isophorone diisocyanate, and toluene-2,4-diisocyanate. By the reaction, polyurethanes (7 or 6) may be formed.

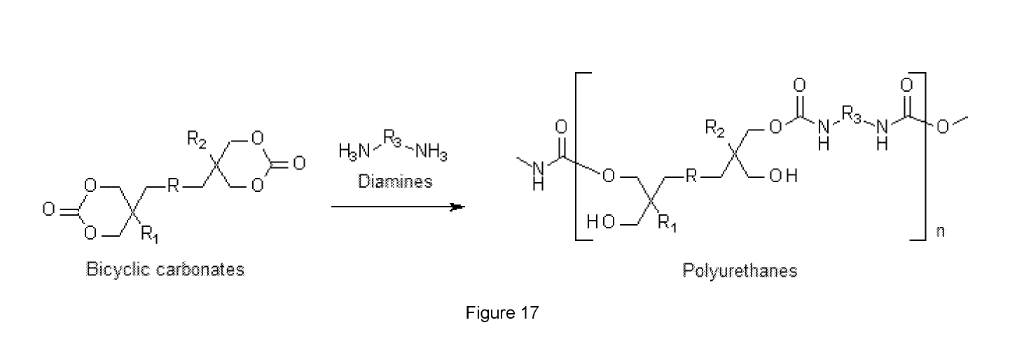

One object of the present invention is to provide dicyclic carbonates having six-membered rings, and manufacturing polyurethanes and copolymers thereof, preferably without the use of phosgene or isocyanate. Below reference is made to FIG. 17.

The six-membered rings of the bicyclic carbonates may originate from polyols such as di-trimethylolpropane (diTMP), di-trimethylolethane (diTME) and derivatives thereof.

In the dicyclic carbonate (diCC), cyclic carbonate group reacts with alkyl and aromatic amine, and diamine compounds by ring opening reaction to produce urethane and diurethane bonds, respectively. The resulting polyurethanes may be produced by an isocyanate-free route. This embodiment provides a facile, green and cost effective production method for polyurethanes from dicyclic carbonates, and coating application.

This embodiment is directed to a method of manufacturing polyurethanes without using phosgene and isocyanate (FIG. 17). Said polyurethanes may then be used for coating applications. Dicyclic carbonate (e.g. diTMP-diCC) may be reacted with diamine compounds in the absence or presence of solvent. Dicyclic carbonate may be reacted with diamine compounds in the absence or presence of catalyst.

According to one embodiment, if no solvent is used also no catalyst is used. Diamino compounds may be chosen from alkyldiamines, e.g. chosen from 1,6-hexamethylenediamine, 1,2-diethylenediamine and isophorone diamine; and phenyl alkyldiamines, e.f. chosen from xylylenediamine. By the reaction between dicyclic carbonate and diamine compounds, polyurethanes may be formed. The molar ratio of used diCC to diamines is not limited. The ratio diCC to diamines may preferably be chosen from a ratio of 10 to 500 wt % such as 10, 50, 100, 250 or 500 wt %, or even more preferred 50 to 200 wt %.

Coating formulated using the mentioned polyurethanes may be cured at ambient temperature (room temperature, RT), or at temperature ranging from ambient to 150° C., preferably ambient to 110° C. General additives such as hardener, softener, catalyst, pigment and binder can be used on the coating application.

In one embodiment the molar ratio of used DiTMP-diCC to diamines is not limited. But the ratio can preferably be used at a ratio of 10 to 500 wt % such as 10, 50, 100, 250 and 500 wt %, or even more preferred 50 to 200 wt %. DiTMP-diCC is solid with 104-106° C. of melting point. Such coating could be cured at ambient temperature (room temperature, RT), or at temperature ranging from ambient to 150° C., preferably ambient to 110° C.

General additives such as hardener, softener, catalyst, pigment and binder can be used on the coating application.

4.1 Ring Opening Reaction of diTMP Bicyclic Carbonate with Diamines and Coating Application in Solvent Free Condition

To obtain homogeneous mixture of said bicyclic carbonate with diamines in a solvent free condition, diCC may be melted.

If diTMP bicyclic carbonate is used, it may be melted at higher than 104° C. to obtain a homogeneous mixture of diTMP bicyclic carbonate with diamines in solvent free condition. However DiTMP-diCC may be melted with diamines at even lower temperature such as 80, 90, or 100° C.

The polymerizations were carried out quickly after mixing and optional melting.

A coating may be applied to a desired substrate surface such as glass, wood, plastic, concrete or ceramic by conventional means. The homogeneous mixture was applied to form the film on the surface of substrate. Curing temperature may be varied depending on the substrate and the curing time may be varied depending on the cure temperature and substrate. The reaction time may be at least a few seconds, e.g. at least 5 seconds, at least 1 minute, at least 1 hour, at least 1 day, or at least 10 days.

4.2 Ring Opening Reaction of diTMP Bicyclic Carbonate with Diamines and Coating Application in Solvent Condition

Dicyclic carbonate (e.g. diTMP-diCC) may be reacted with diamine compounds in solvent without catalyst. The reaction and application may be performed in solution form and any organic solvent may be used. However, preferred solvents are alcohols (e.g. methanol, ethanol and propanol), (cyclic) ethers (e.g. diethyl ether and THF), ketones (e.g. acetone, ethylmethylketone), toluene, acetonitrile, halogenated alkane (dichloromethane and chloroform), dimethylformamide, and pyridine or mixtures of the same or mixtures containing said solvents. Use of solvent provides the homogenization, polymerization and coating application of dicyclic carbonate (e.g. diTMP bicyclic carbonate) with diamines at lower temperature. The solubility of dicyclic carbonate (e.g. diTMP-diCC) in most solvents is low for general coating application. Typical solvent content is 0-65% depending on coating types. Meanwhile it has been observed that diamines enhanced the solubility of dicyclic carbonate (e.g. DiTMP-diCC) in solvents at lower temperature. The ratio of used solvent to dicyclic carbonate (e.g. DiTMP-diCC) is not limited. But the ratio can preferably be 1 to 500 wt % such as 1, 10, 50, 100, 250 and 500 wt %, or even more preferred 20 to 200 wt %. The ratio may be varied depending on application methods such as spray, brush and roll.

Some solvents such as acetonitrile, dimethyformaide and dichloromethane show good solubility of dicyclic carbonate, such as diTMPdiCC, at ratio of 1 to 1 with diamines in RT. Also the reaction takes place at RT, thus the mixture may be applied, cured and dried on the surface at RT or higher temperature.

Some solvents such as ethanol, THF, 2-propanol and show partial solubility at 60° C., but with reaction the solution became homogenized within 5 min at 60° C. The solution may be applied, cured and dried on the surface at 60° C. or higher temperature. Additionally, after reaction for certain time (1-5 min) at 60° C., the solution may be applied, cured, and dried on the surface at RT.

### EXAMPLES

The present invention is further explained in more detail with reference to the following examples. These examples, however, should not be interpreted as limiting the scope of the present invention in any manner.

### Analyses and Structure Elucidation

Quantitative analyses of reaction components was performed using gas chromatography (GC, Varian 430-GC, Varian, USA) equipped with FactorFour Capillary column, VF-1 ms (Varian, 15M×0.25 mm) and a flame ionization detector. The initial column oven temperature was increased from 50 to 250° C. at a rate of 20° C./min. After removing the solid portion by centrifugation or filtration, the samples diluted with acetonitrile to a final concentration of 0.1-0.5 mg/mL, were injected in split injection mode of 10% at 275° C. The conversion of substrates and ratio of products were calculated by comparison of peak areas on the gas chromatograms.

The structures of the products were then determined by ¹H and ¹³C-NMR using 400 MHz NMR (Bruker, UltraShield Plus 400, Germany). FT-IR analyses were performed using Nicolet-iS5 (Thermo Scientific, USA).

### Determination of Physical Properties for the Applied Coating

For coatings in accordance with the present invention, the pencil hardness of the coating films may be measured by following ASTM D 3363 (2005) using pencils with leads ranging in hardness from 4B to 4H. An acceptable pencil hardness level for a coating is F or more. Examples of unacceptable levels of hardness are B or less. The apparent transparency of the coating films on the glass surface was determined, and ranged from 1 (low) to 5 (high transparent, colorless). The formation of urethane group from cyclic carbonate was determined from samples collected from coating by FT-IR analysis.

### Synthesis and Reactions of Allylated Polyol Cyclic Carbonate

### Example 1

### Synthesis of Monoallylated TMP Cyclic Carbonate

50 g TMP-ME was dissolved in 800 mL DMC in a 2 L reaction vessel. The reactant solution with 750 g molecular sieves was heated in 120° C. oil bath for 5 hr. TMP-ME cyclic carbonate was obtained in 96.6% yield according to GC, after removal of solid residue and concentration.

### Example 2

### Ring Opening Reaction of TMP-ME Cyclic Carbonate with Amine and Diamine Compounds

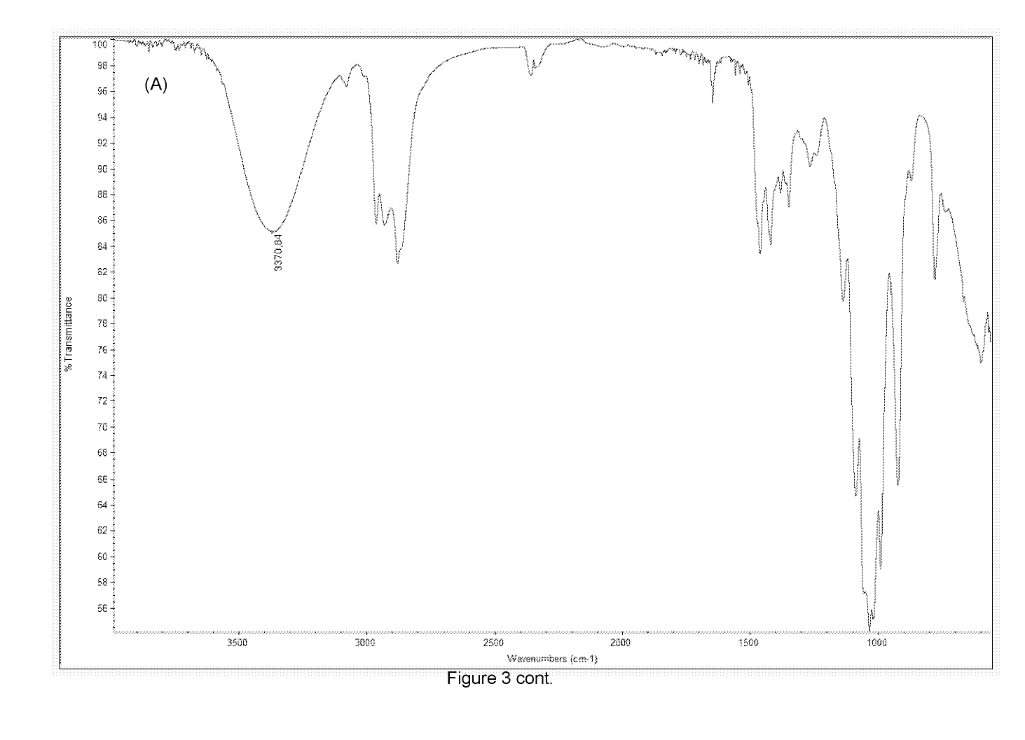

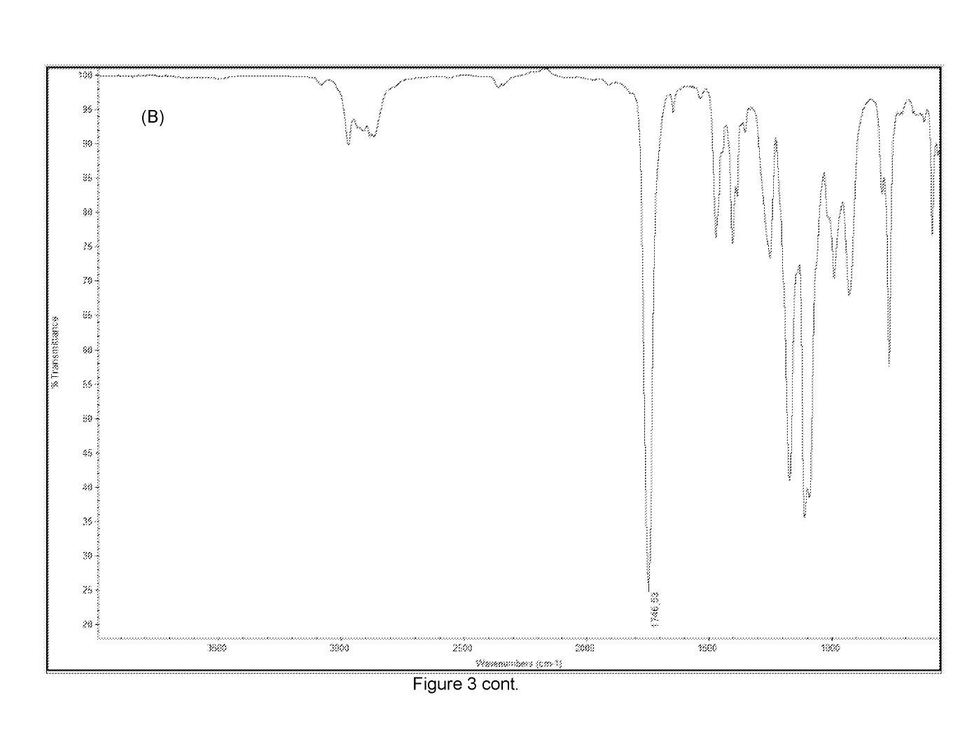

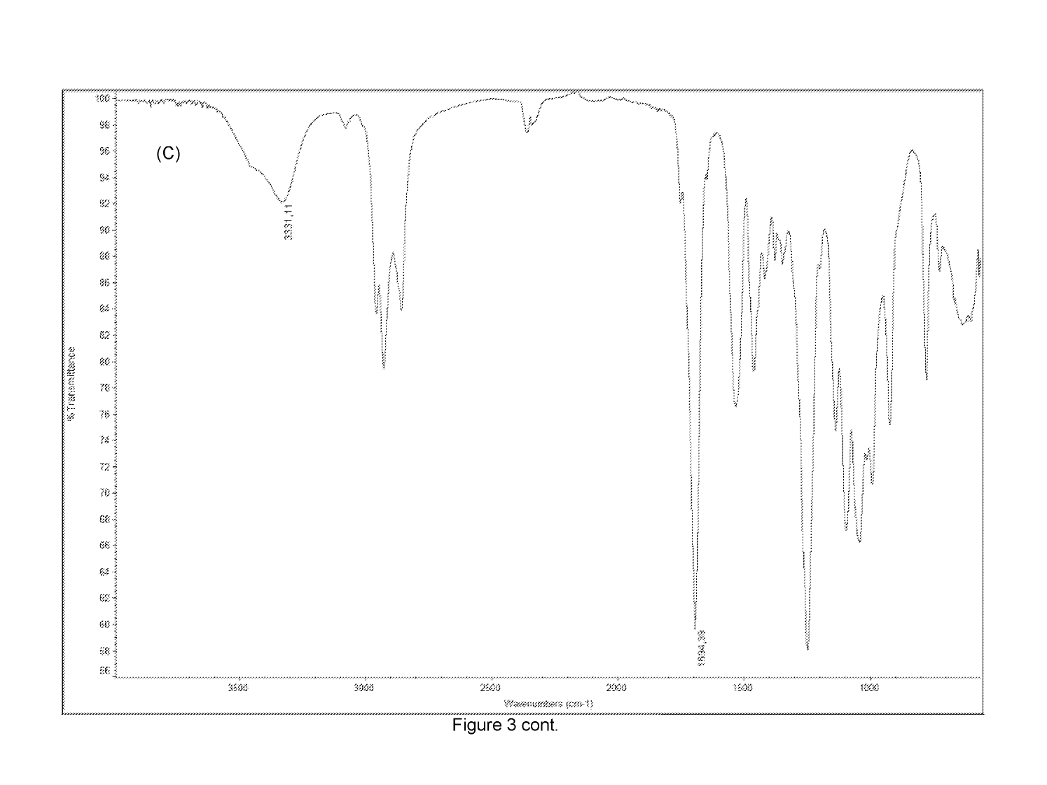

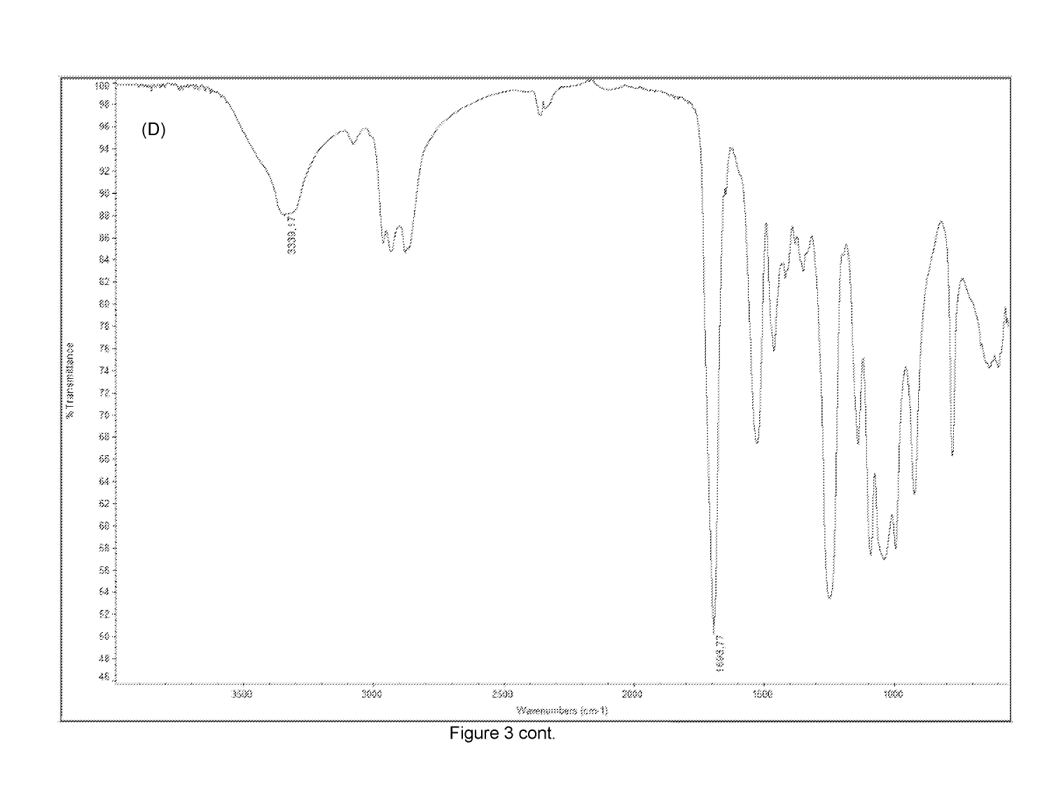

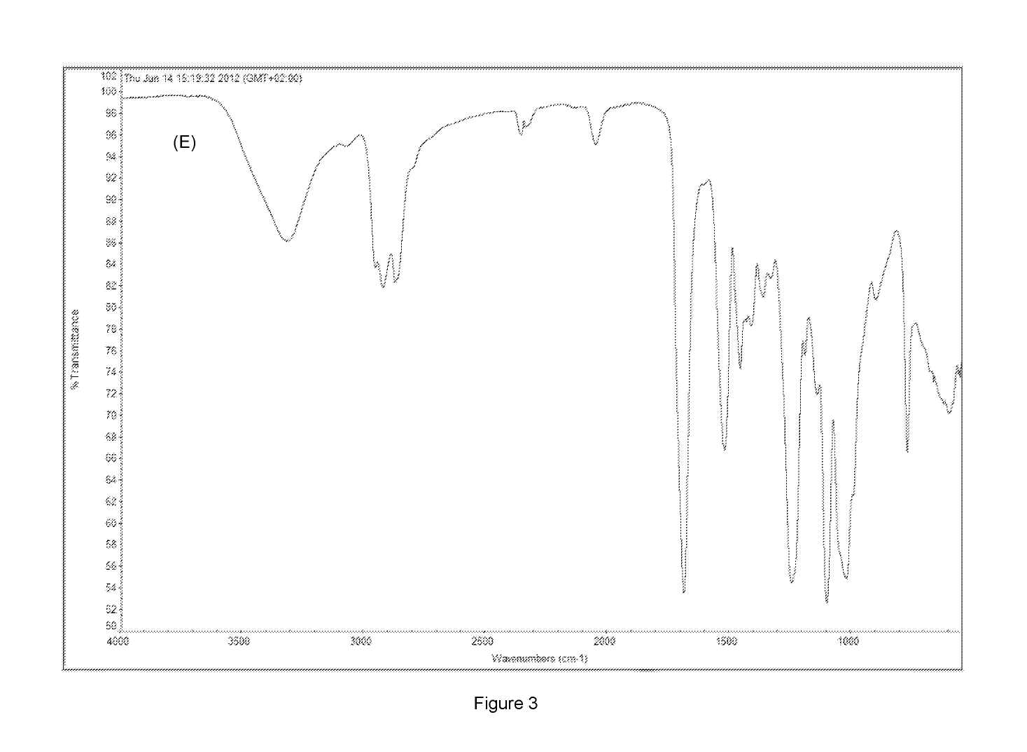

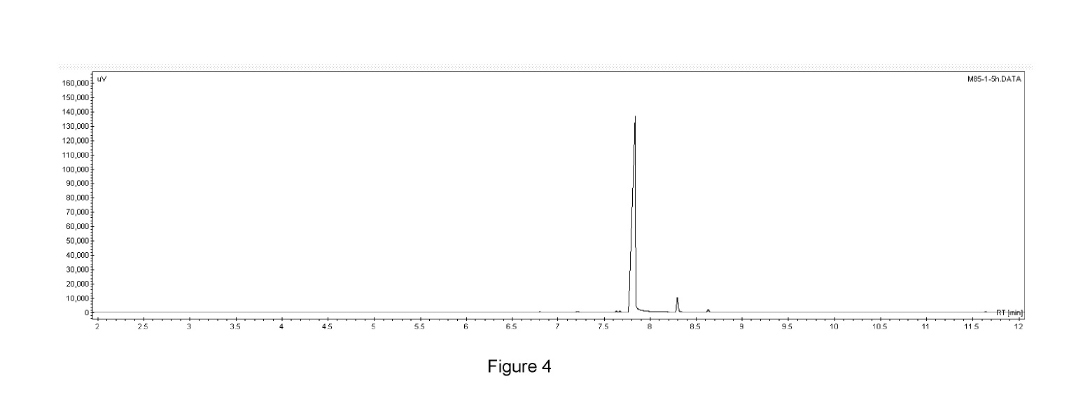

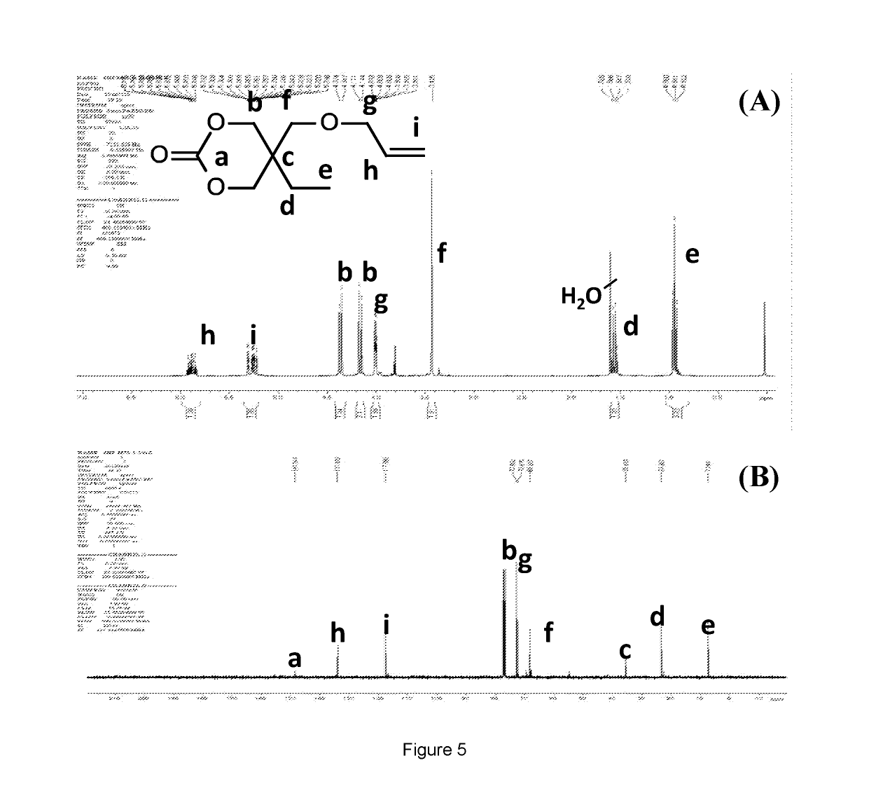

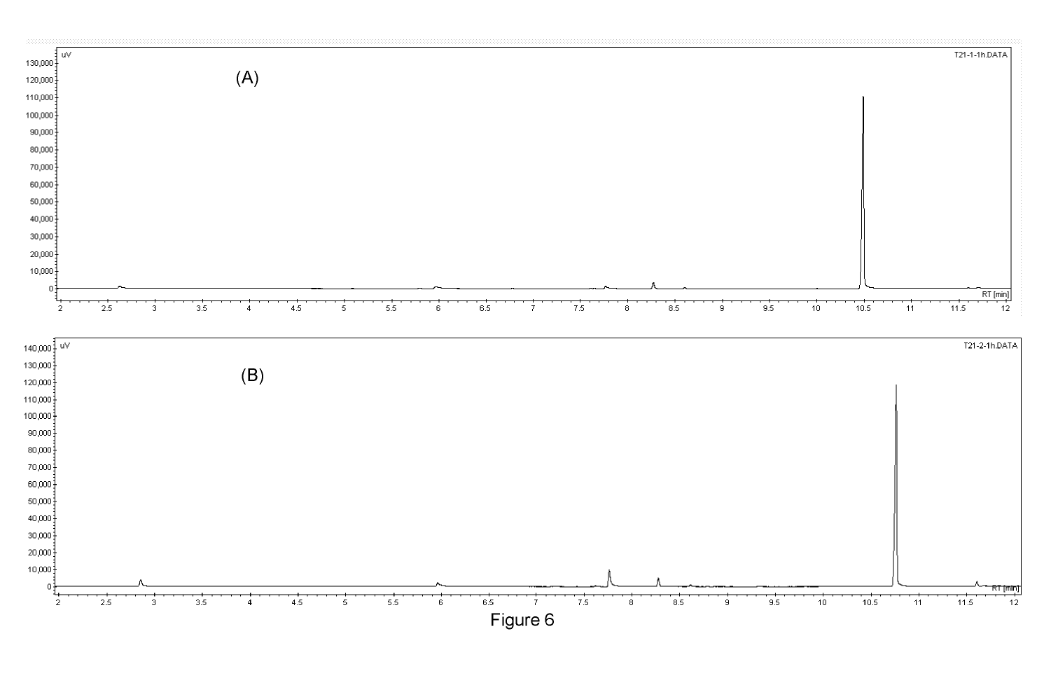

100 mg (0.5 mmol) TMP-ME cyclic carbonate was reacted with 0.55 mmol various amine compounds and 0.28 mmol various diamine compounds in 4 mL vial at 50° C. with or without solvent using Thermomixer. Small aliquots of reaction samples were taken for analysis at varying time intervals. The GC peak of TMP-ME cyclic carbonate in FIG. 4 was shifted to corresponding amine (FIG. 6 (A,B)), and the peaks shifts in FT-IR spectra were observed in FIG. 3(B, C and D).

In FIG. 3 FT-IR spectra show the peak shifts of functional groups in each reaction step. (A) TMPME: the strong broad peak in 3000-3500 cm⁻¹ indicates —OH group. (B) TMPME cyclic carbonate: a new peak at 1750 cm⁻¹ indicates carbonyl group of cyclic carbonate, and the strong broad peak of —OH group in 3000-3500 cm⁻¹ disappeared with formation of cyclic carbonate. (C) Urethane from reaction of TMPME cyclic carbonate and hexylamine: a peak at 1750 cm⁻¹ was shifted to 1700 cm⁻¹, which is an amide (urethane) bond, and a new peak at 3000-3500 cm⁻¹ appeared for —OH group resulting from ring opening of cyclic carbonate. (D) Diurethane from reaction of TMPME cyclic carbonate and ethylenediamine: a peak at 1750 cm⁻¹ in (B) was shifted to 1700 cm⁻¹, which is an amide (urethane) bond, and a new peak at 3000-3500 cm⁻¹ appeared for —OH group resulting from ring opening of cyclic carbonate. (E) Polymer from reaction of (D) with ethanedithiol: a peak at 900 cm⁻¹ in (D), which is C—H of mono-substituted alkene, disappeared by reaction of alkene with thiol group.

TABLE 1

| | TMPME-CC | | Amine | | Reaction | Conversion |
|---|---|---|---|---|---|---|
| Run | (mmol) | Amine | (mmol) | Solvent | time (h) | (%) |
| 1 | 0.5 | n-hexylamine | 0.55 | | 1 | 98.9 |
| 2 | 0.5 | n-hexylamine | 0.55 | ACN, 1 mL | 1 | 74.4 |
| 3 | 0.5 | n-hexylamine | 0.55 | DMSO, 1 mL | 1 | 51.2 |
| 4 | 0.5 | n-hexylamine | 0.55 | DMSO, 1 mL | 15 | 99.6 |
| 5 | 0.5 | n-hexylamine | 0.55 | DMF, 1 mL | 15 | 99 |
| 6 | 0.5 | n-hexylamine | 0.55 | Pyridin, 1 mL | 15 | 98.2 |
| 7 | 0.5 | Cyclohexylamine | 0.55 | | 1 | 91.3 |
| 8 | 0.5 | Dipropylamine | 0.55 | | 24 | 89.9 |
| 9 | 0.5 | Ethylenediamine | 0.28 | | 45 | 85 |
| 10 | 0.5 | 1,6-NMDA | 0.28 | | 45 | 90 |
| 11 | 0.5 | Isophoronediamine | 0.28 | | 45 | 95 |

### Example 3

### Reaction or Polymerization of Urethane and Di-Urethane Products Obtained in Example 2

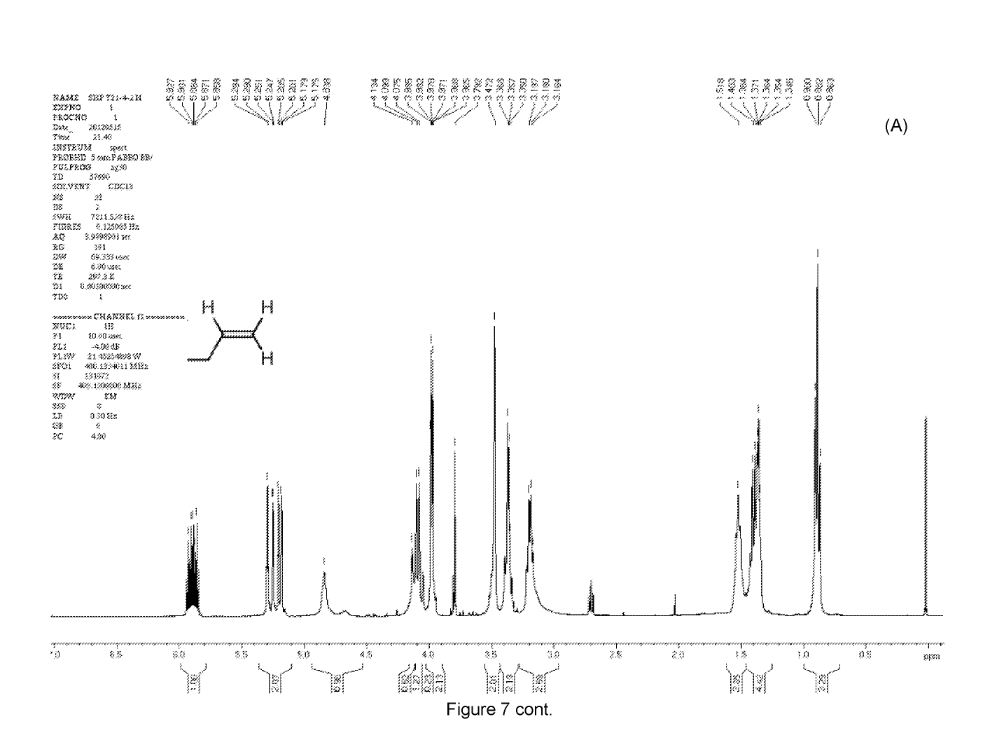

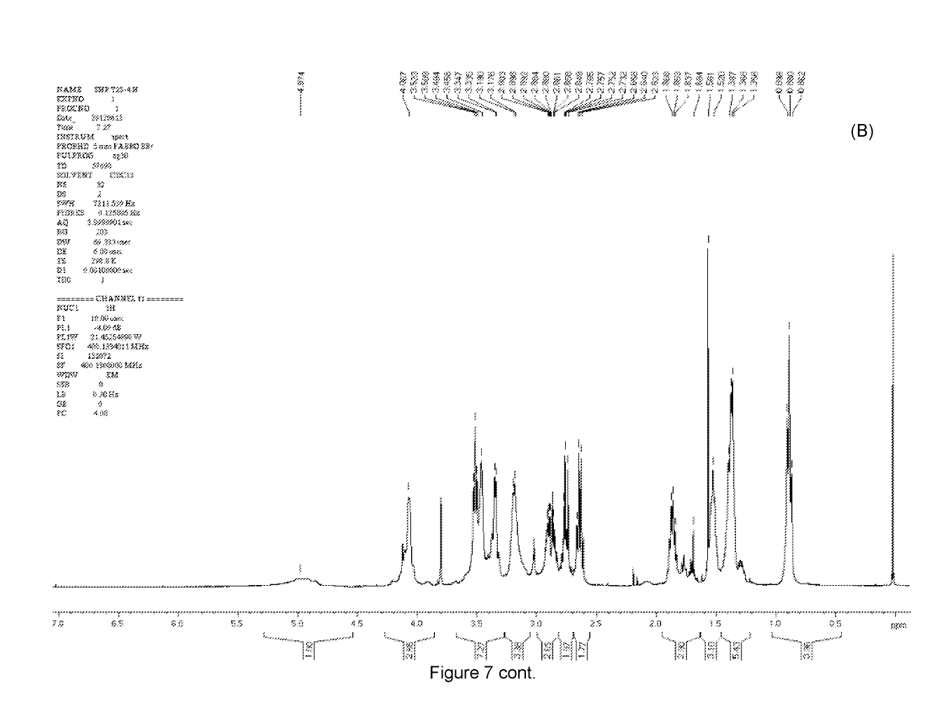

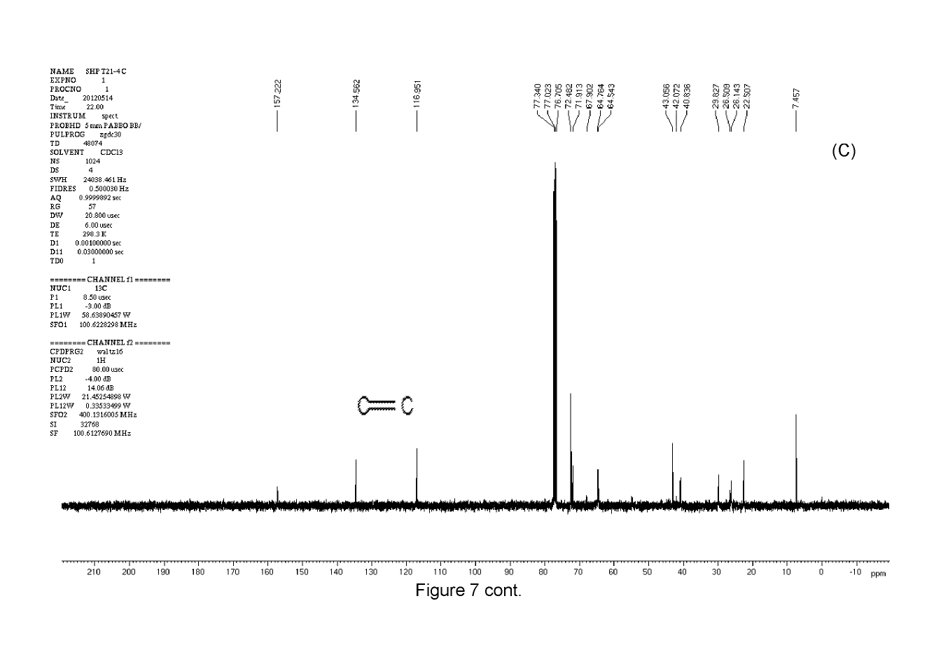

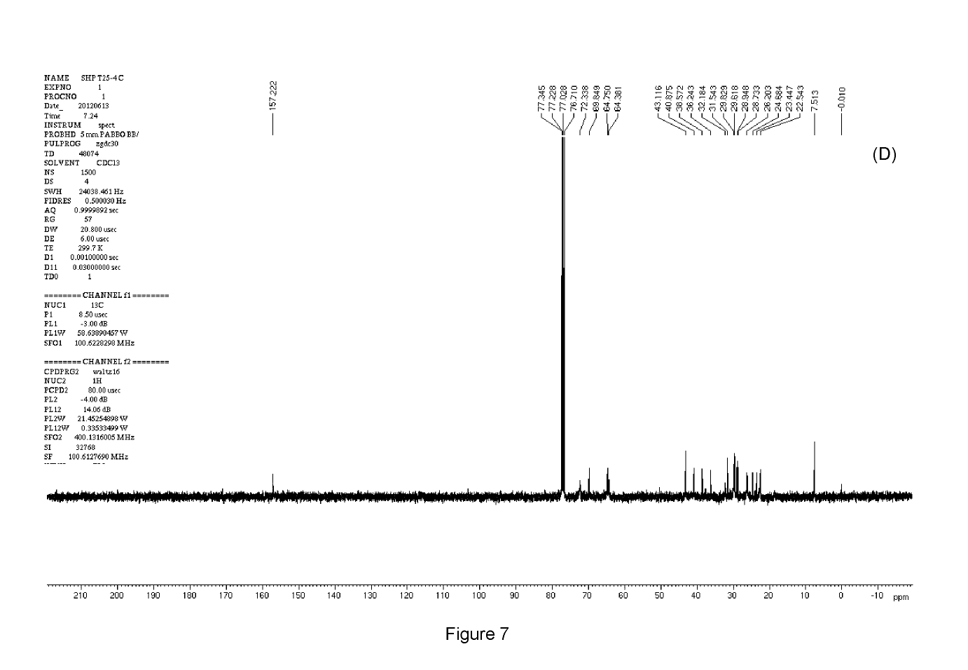

The reactions were allowed for longer time (1-5 days) to reach over 97% conversion in example 2. Resulting products were reacted in 0.4 mL CDCl₃ with 1,2-ethylendithiol and 1% (w/w) AIBN as an initiator in 80° C. for 15 hr. The resulting products were analyzed by NMR, and the conversion yield was estimated by 1H-NMR. The reactions take place between allyl group and thiol group by thermal reaction with AIBN. After reaction, the allyl group disappeared completely to form new C—S bond in ¹H and ¹³C-NMR (see FIG. 7).

TABLE 2

| | | | 1,2-Ethane- | Conver- | Product |
|---|---|---|---|---|---|
| | Substrate (25 mg) | | dithiol | sion | 5 or 6 |
| Run | 3 or 4 in FIG. 2 | mmol | (mmol) | (%) | FIG. 2( |
| 1 | 3a (R = hexyl) | 0.083 | 0.042 | 98 | 5a |
| 2 | 3b (R = cyclohexyl) | 0.084 | 0.042 | 76 | 5b |
| 3 | 3c (R = dipropyl) | 0.083 | 0.042 | 99 | 5c |
| 4 | 4a (R1 = ethylene) | 0.096 | 0.096 | 99 | 6a |
| 5 | 4b (R1 = hexamethylene) | 0.079 | 0.079 | 99 | 6b |
| 6 | 4c (R1 = isophorine) | 0.067 | 0.067 | 99 | 6c |
| 7 | 4d (R1 = m-xylylene) | 0.074 | 0.074 | 99 | 6d |

### Part 2. Synthesis and Polymerization of Acrylated or Methacrylated Polyol Cyclic Carbonates

### Example 4

### Synthesis of Methacrylated TMP

10 g TMP was dissolved in 100 mL methyl methacrylate in a 250 mL reaction vessel with magnetic stirring in oil bath at 60° C. The reaction was started by addition of 1 g N435 along with 40 g molecular sieves. Aliquots were withdrawn at different time intervals for analysis of the reaction components. After 24 hr reaction, 60% (GC) TMP-mMA and 22% TMP-dimethacrylate (TMP-diMA) were obtained with 82% TMP conversion. TMP-mMA and TMP-diMA were purified by column (5×25 cm) silica flash chromatography using ethyl acetate and mixture of ethylacetale and methanol (1:1) as eluent.

### Example 5

### Synthesis of Methacrylated TMP Cyclic Carbonate

Purified TMP-mMA was converted to the corresponding six-membered cyclic carbonate. 1.5 g TMP-mMA was dissolved in 50 mL DMC in a 250 mL reaction vessel. The reactant solution with 20 g molecular sieves was heated in 120° C. oil bath for 20 hr. TMP-mMA cyclic carbonate was obtained at 96% yield according to GC.

### Example 6

### Ring Opening Reaction of TMP-mMA Cyclic Carbonate with Amine and Diamine Compounds

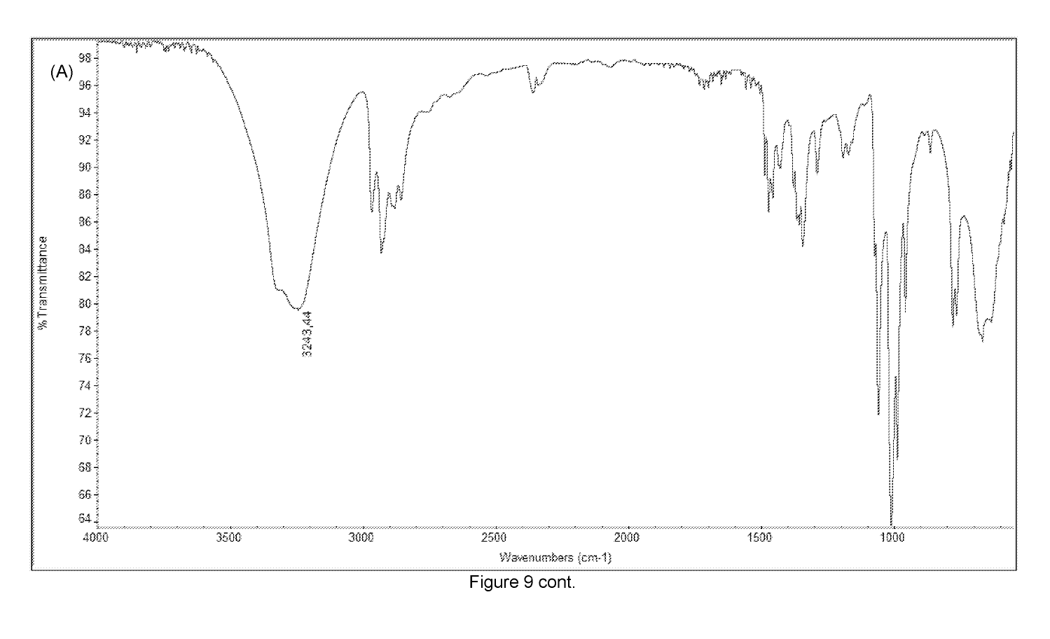

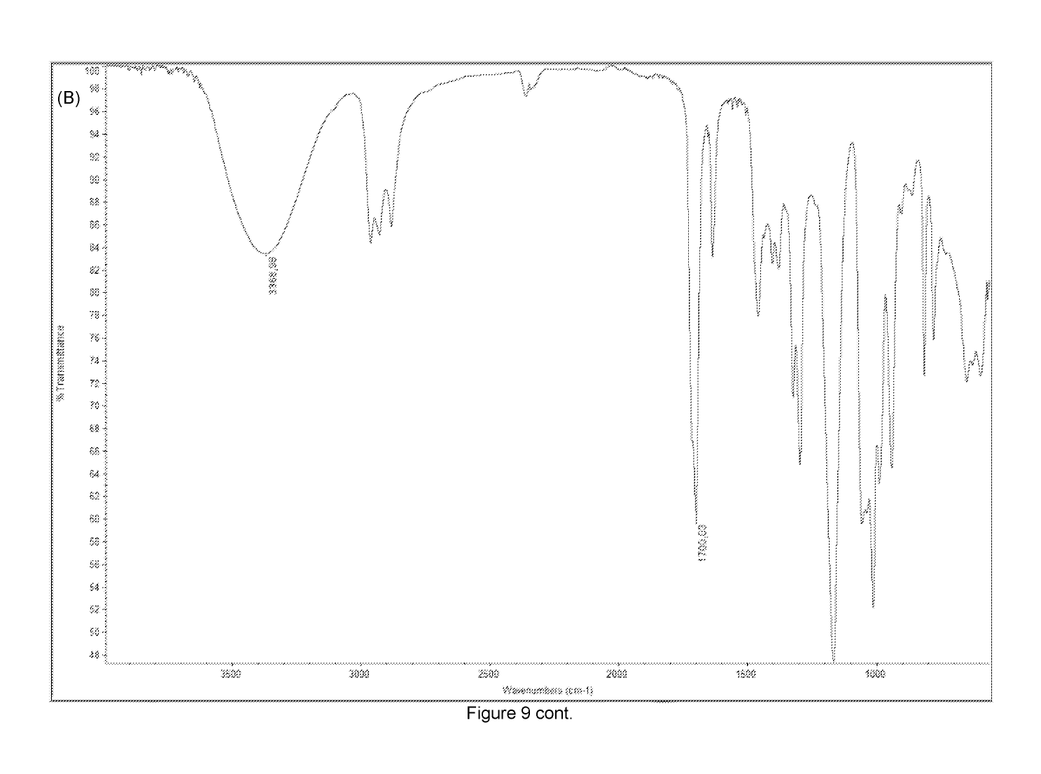

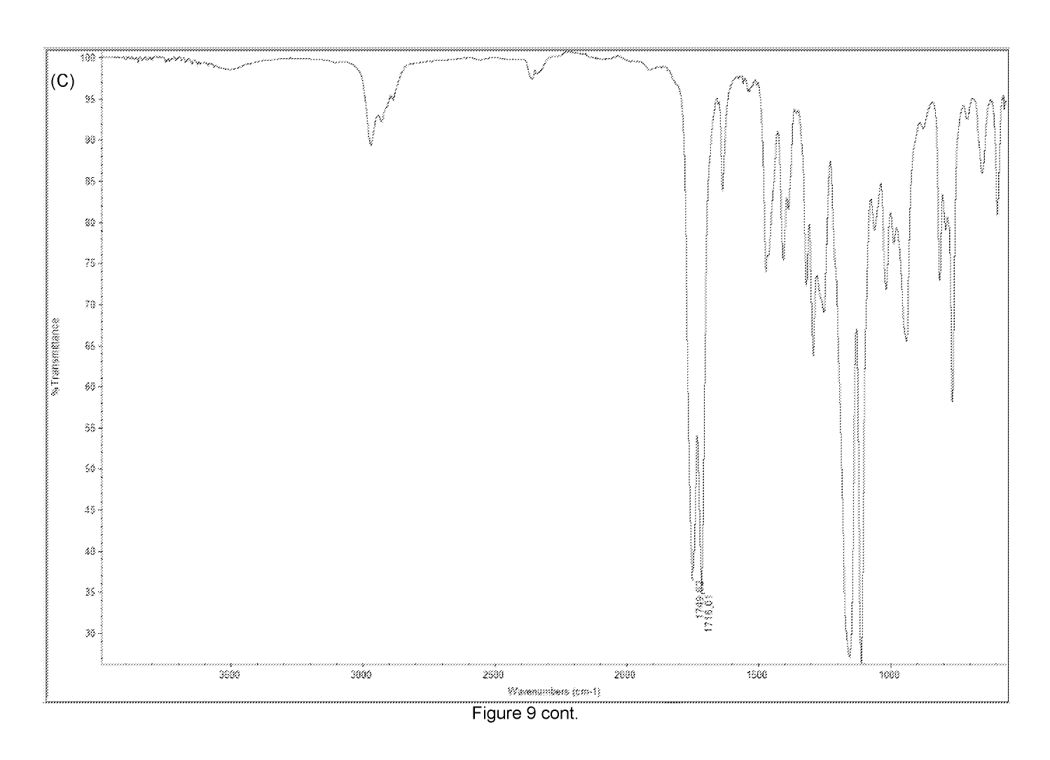

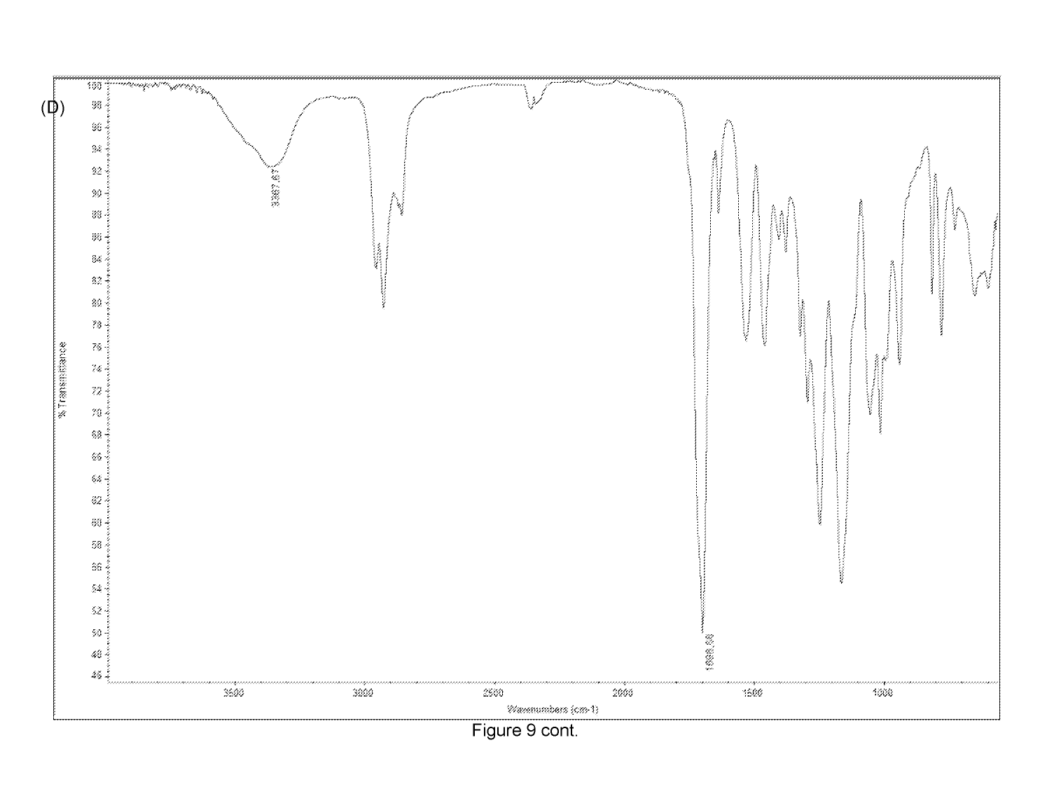

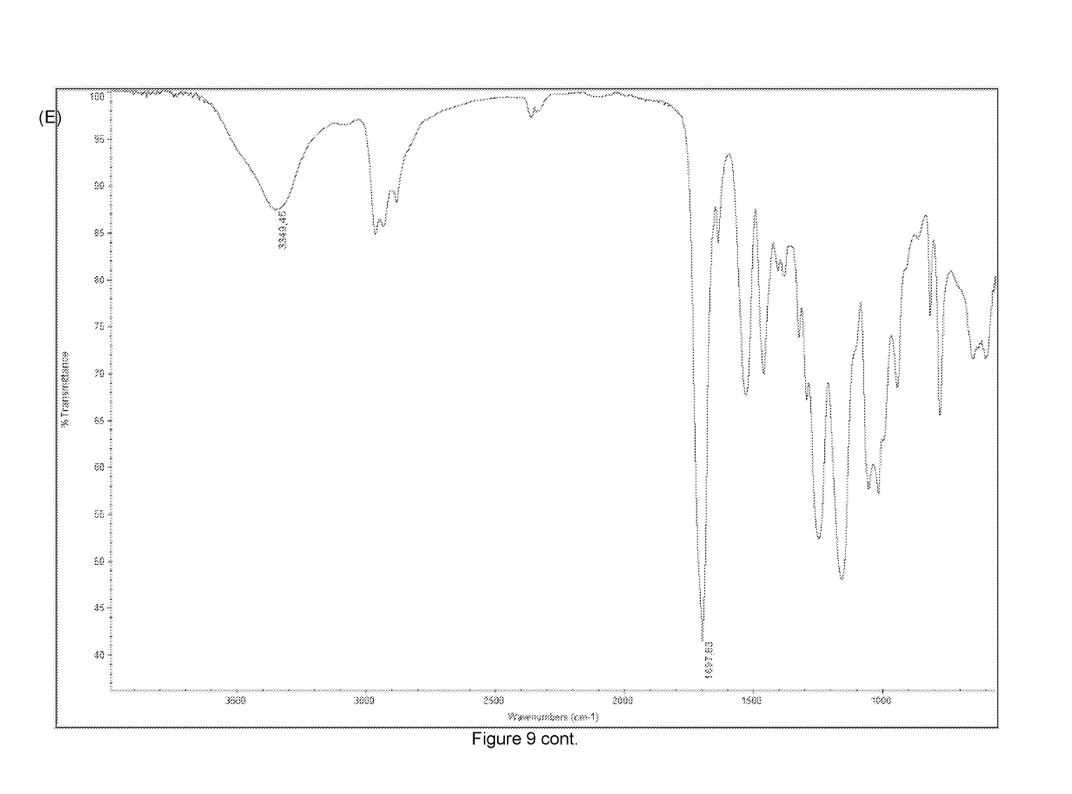

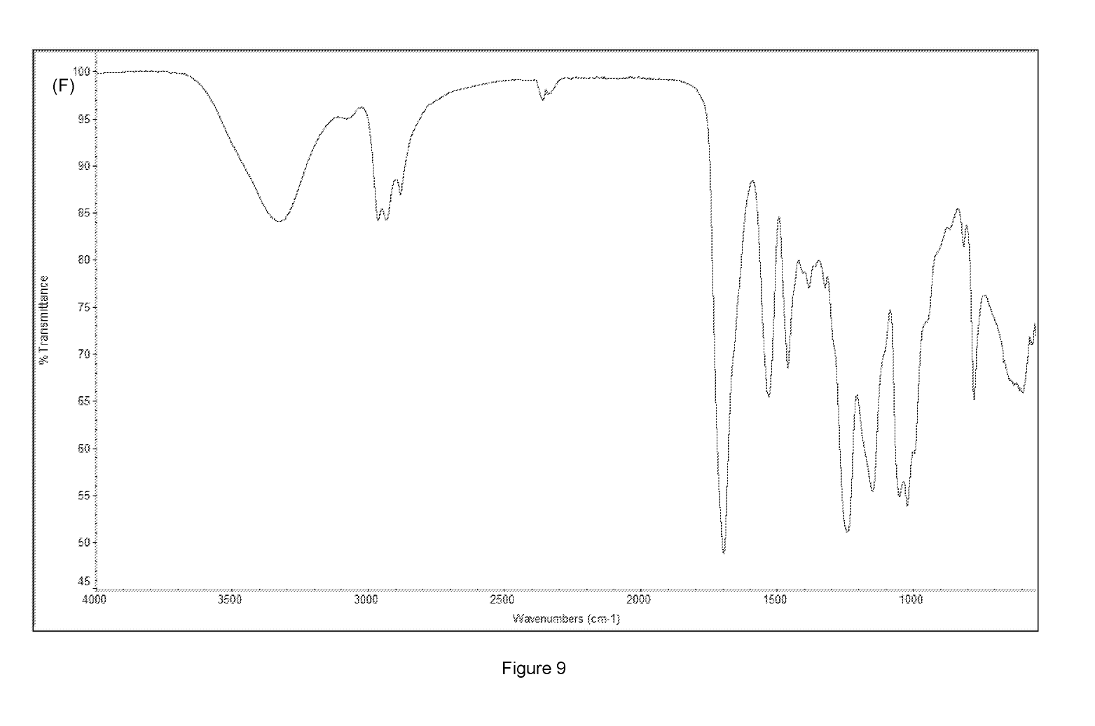

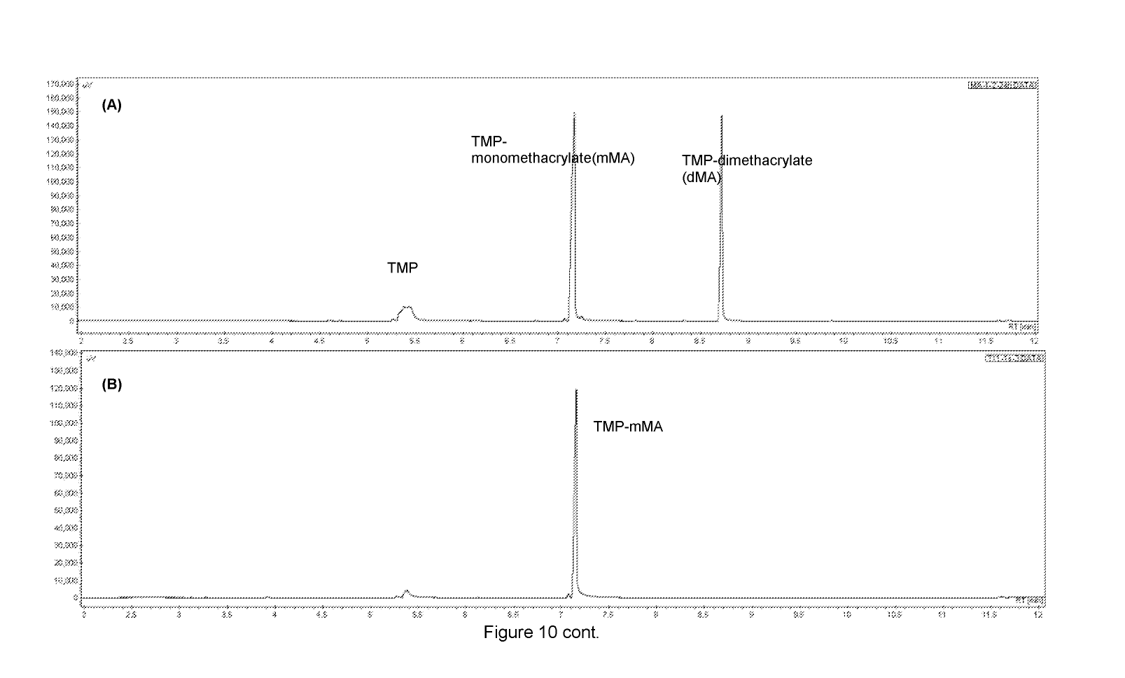

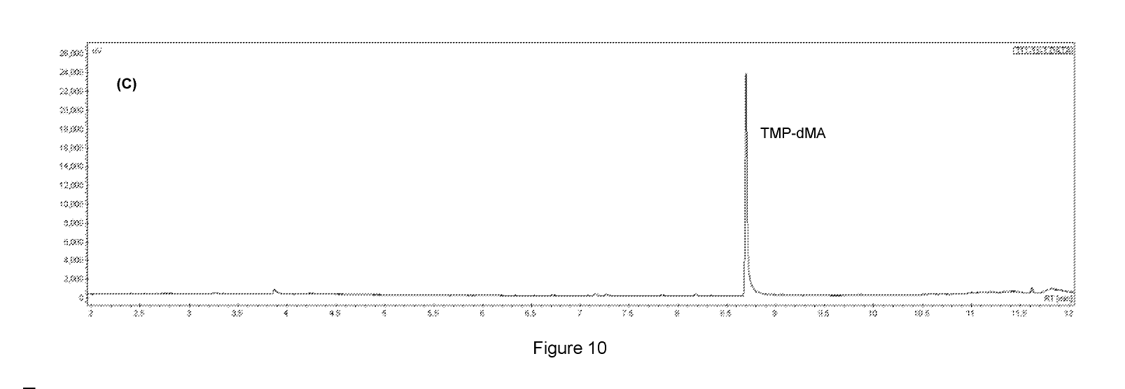

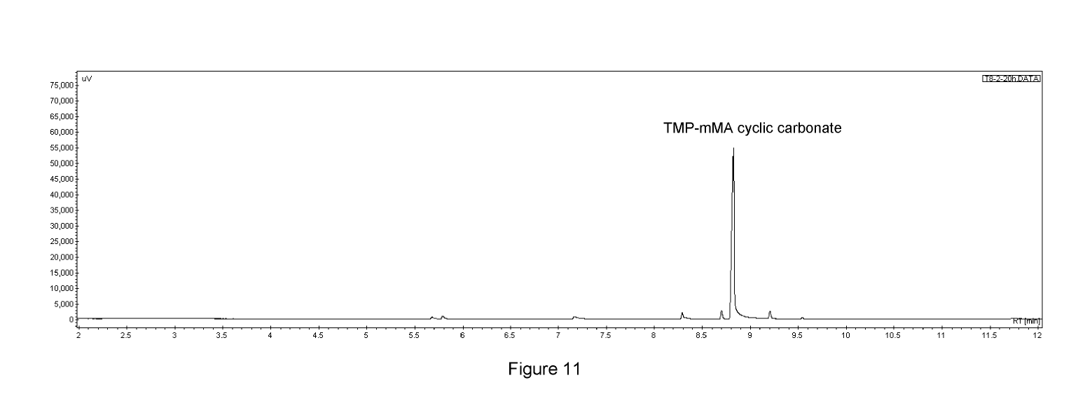

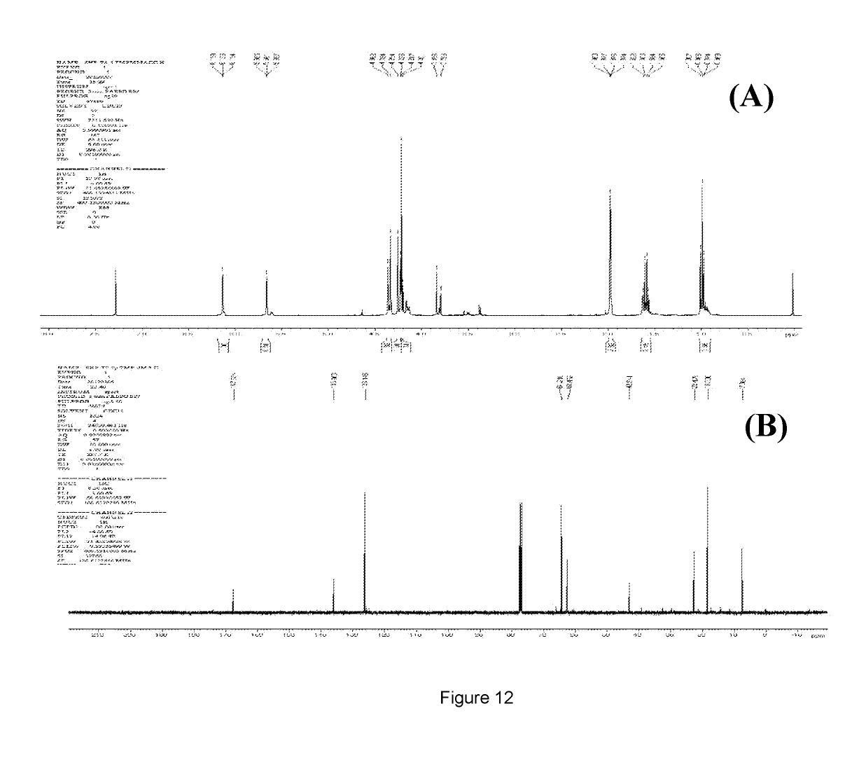

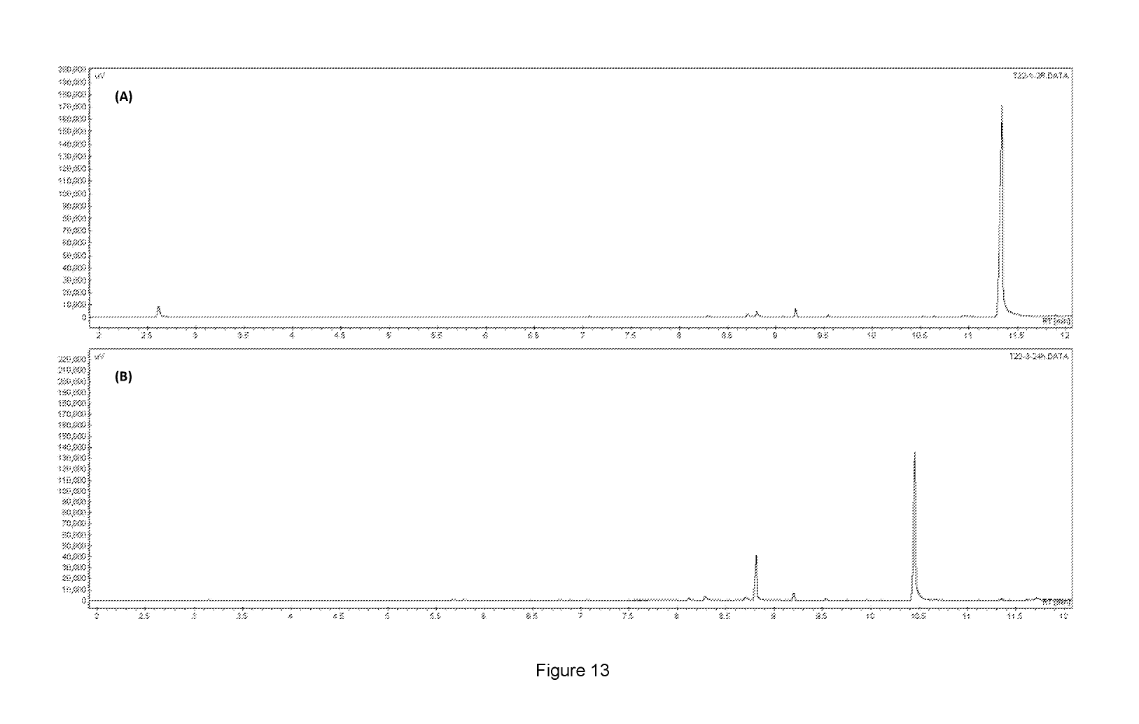

50 mg (0.22 mmol) TMP-mME cyclic carbonate was reacted with 0.25 mmol of various amine compounds or 0.11 mmol various diamine compounds in 4 mL vial at 50° C. with or without solvent using Thermomixer as shown in Table. Small aliquots of reaction samples were taken for analysis at varying time intervals. The GC peak of TMP-mMA cyclic carbonate in FIG. 11 was shifted to corresponding amine (FIG. 13 (A,B)), and the peaks shifts in FT-IR spectra were observed in FIG. 9(C, D and E).

In FIG. 9 the FT-IR spectra show the peak shift of functional groups in each reaction step. (A) TMP: the strong broad peak in 3000-3500 cm⁻¹ indicates —OH group. (B) TMP-mMA: a new peak at 1700 cm⁻¹ indicates carbonyl group of methacrylate. (C) TMP-mMA cyclic carbonate: a new peak at 1750 cm-1 indicates carbonyl group of cyclic carbonate, and the strong broad peak of —OH group in 3000-3500 cm⁻¹ disappeared with formation of cyclic carbonate. (D) Urethane from reaction of TMP-mMA cyclic carbonate and hexylamine: a peak at 1750 cm⁻¹ in (C) disappeared with formation of amide (urethane) bond, which was overlapped at 1700 cm⁻¹, and a new peak at 3000-3500 cm⁻¹ appeared for —OH group resulting from ring opening of cyclic carbonate. (E) Diurethane from reaction of TMP-mMA cyclic carbonate and ethylenediamine: a peak at 1750 cm⁻¹ in (C) disappeared with formation of amide (urethane) bond, which was overlapped at 1700 cm⁻¹, and a new peak at 3000-3500 cm⁻¹ appeared for —OH group resulting from ring opening of cyclic carbonate. (F) Polymer from reaction of (E) by thermal polymerization: a peak at 900 cm⁻¹ in (E), which is C—H of mono-substituted alkene, disappeared by new C—C bond formation on polymerization of methacrylate.

TABLE 3

| | TMPmMA-CC | | Amine | Solvent | Reaction | Conversion |
|---|---|---|---|---|---|---|
| Run | (mmol) | Amine | (mmol) | mL | time (h) | (%) |
| 1 | 0.22 | n-hexylamine | 0.25 | — | 0.5 | 94.3 |
| 2 | 0.22 | n-hexylamine | 0.25 | — | 2 | 97 |
| 3 | 0.22 | Cyclohexylamine | 0.25 | — | 0.5 | 85.5 |
| 4 | 0.22 | Cyclohexylamine | 0.25 | — | 5 | 96.2 |
| 5 | 0.22 | Dipropylamine | 0.25 | — | 24 | 79.1 |
| 6 | 0.22 | Ethylenediamine | 0.11 | — | 24 | 69 |
| 7 | 0.22 | Ethylenediamine | 0.11 | DMSO, 1 mL | 24 | 76 |
| 8 | 0.22 | Isophoronediamine | 0.11 | — | 24 | 75 |
| 9 | 0.22 | Isophoronediamine | 0.11 | — | 120 | 96 |

### Example 7

### Polymerization of Urethane and Di-Urethane Products Obtained in Example 6

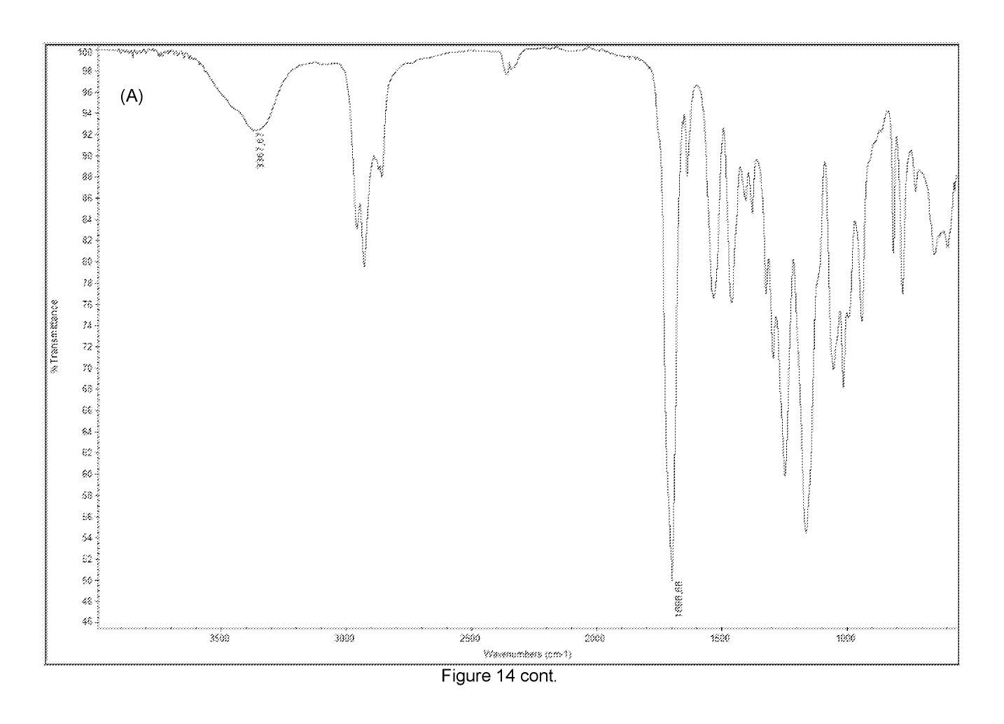

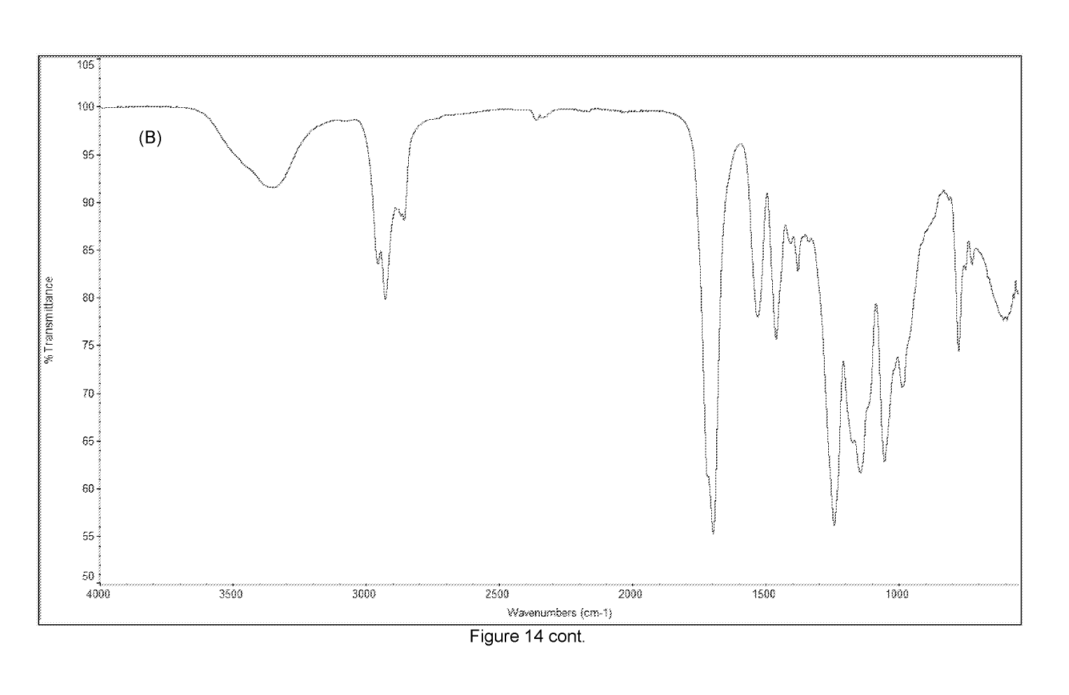

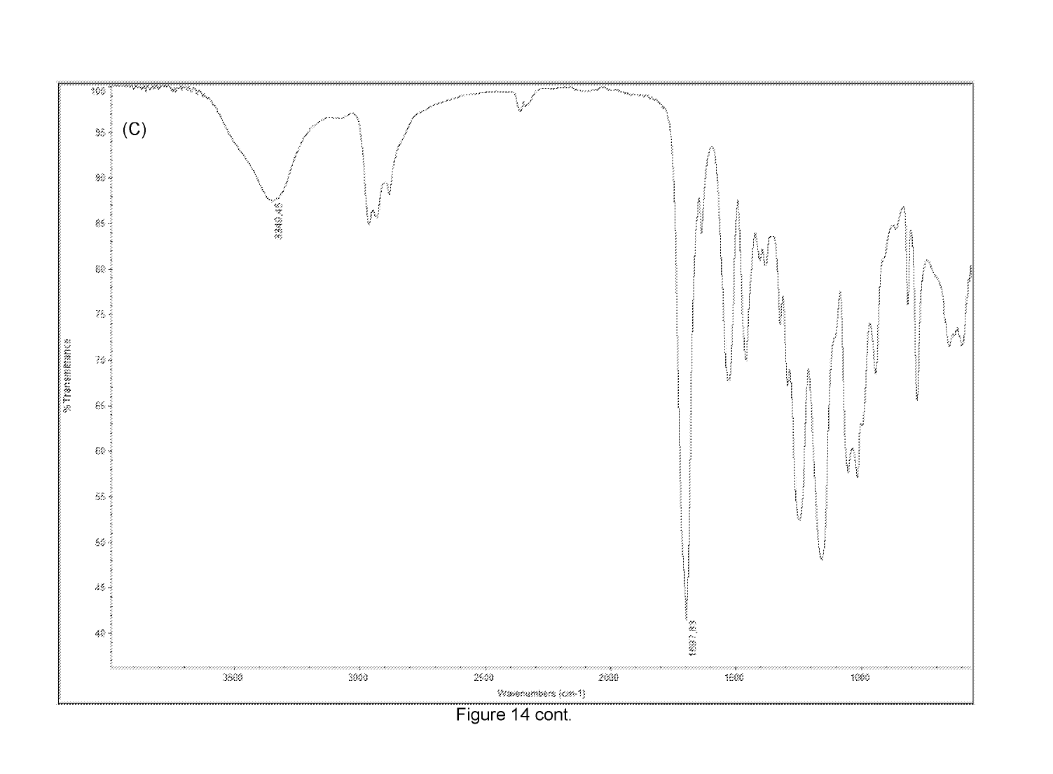

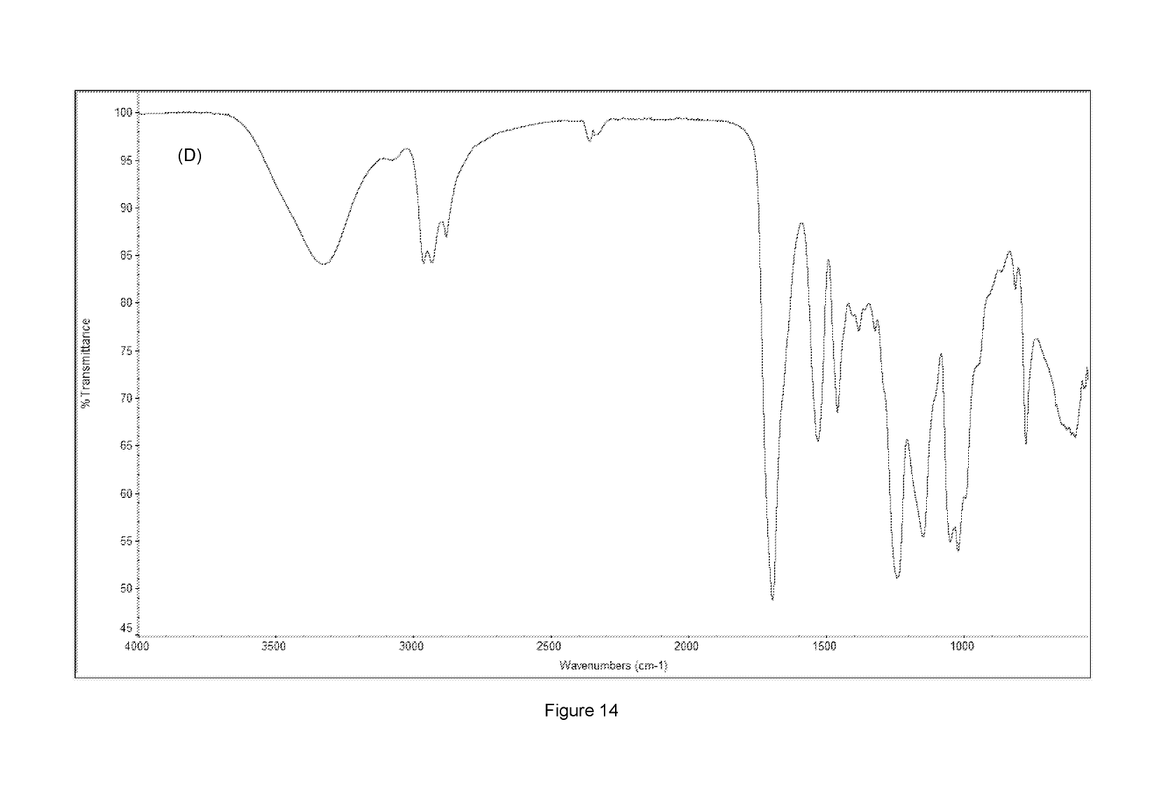

The reactions were allowed for longer time (1-5 days) to reach over 97% conversion in example 6. The resulting products (25 mg) were reacted in 0.1 mL CDCl₃ with 1% (w/w) AIBN as an initiator at 110° C., and CDCl₃ was evaporated during the reaction. Small aliquots of reaction samples were taken for analysis at varying time intervals. Resulting products was analyzed and conversion yield was estimated by FT-IR. The reactions take place in α,β-unsaturated double bond of methacryl group by thermal reaction with AIBN. After reaction, the α,β-unsaturated double bond disappeared in FT-IR to form new C—C bond (FIG. 14).

TABLE 4

| | | Reaction | Conver- | Product |
|---|---|---|---|---|
| | Substrate (25 mg) | Time | sion | 13 or 14 |
| Run | 11 or 12 FIG. 8 | (h) | (%) | FIG. 8 |
| 1 | 12a (R = n-hexyl) | 24 | 99 | 14a |
| 2 | 12b (R = cyclohexyl) | 24 | 70 | 14b |
| 3 | 12c (R = dipropyl) | 24 | 99 | 14c |
| 4 | 11a (R1 = ethylene) | 5 | 57 | 13a |
| 5 | 11a (R1 = ethylene) | 24 | 85 | 13b |
| 6 | 11b (R1 = Isophorone) | 24 | 60 | 13c |

### Part 3. Synthesis and Reactions of Hydroxyl Cyclic Carbonate Example 8. Synthesis of TMP Cyclic Carbonate

50 g TMP was dissolved in 800 mL DMC in a 2 L reaction vessel. The reactant solution with 750 g molecular sieves was heated in 120° C. oil bath for 5 hr. TMP cyclic carbonate was obtained in 97% purity after purification by silica chromatography.

### Example 9

### Ring Opening Reaction of TMP Cyclic Carbonate with Diamine Compounds and Polymerization of Resulting Diurethane Compounds

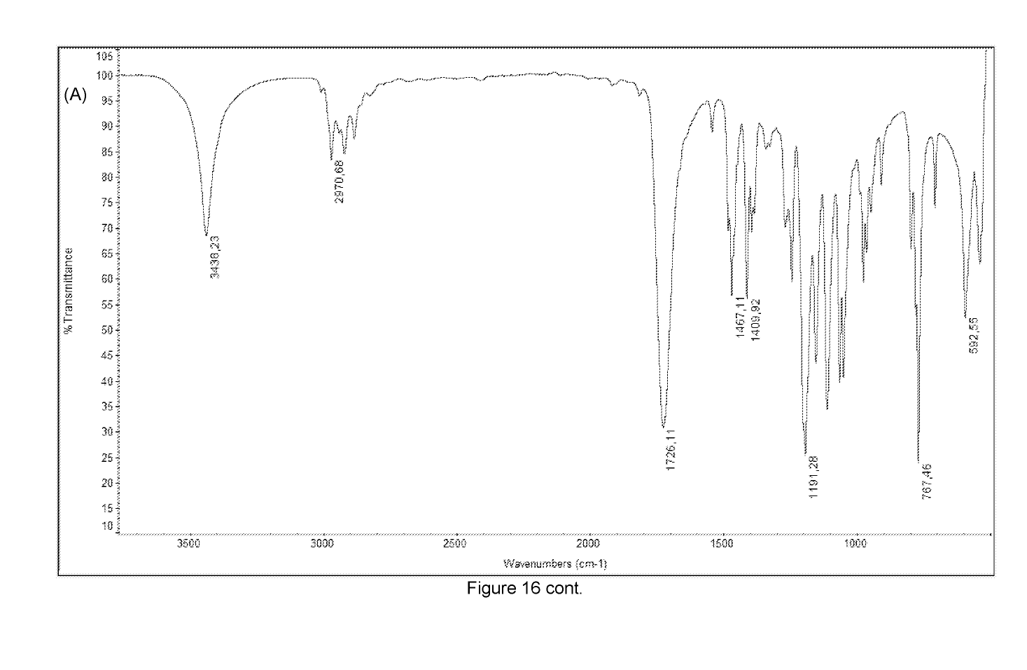

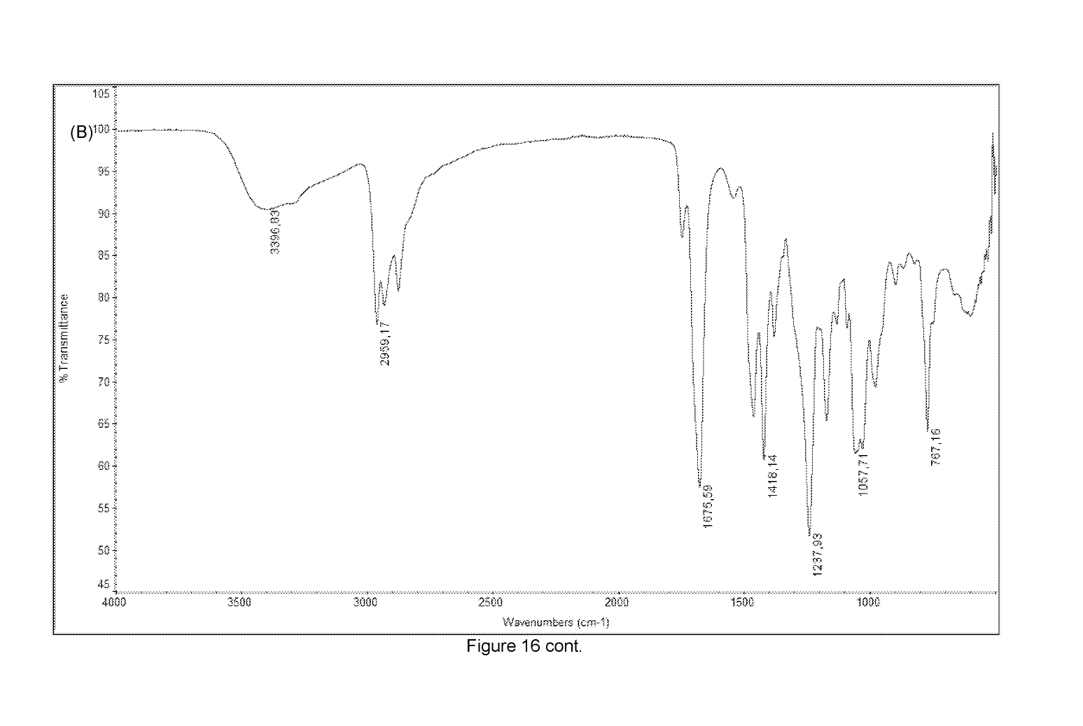

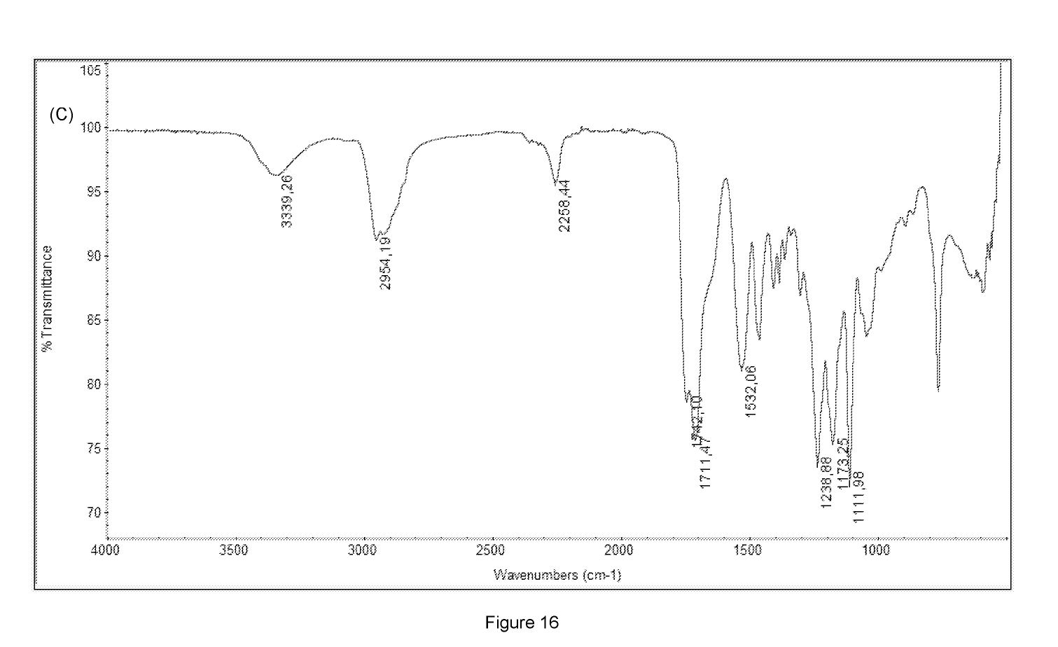

35 mg (0.2 mmol) TMP cyclic carbonate was reacted with 20.3 mg (0.14 mmol) N,N′-di-n-propylethylenediamine in 4 mL vial at 70° C. without solvent using Thermomixer for 6 hr. Small aliquots of reaction samples were taken for analysis at varying time intervals. The peaks shifts in FT-IR spectra were observed in FIG. 16(A to B). Resulting diurethane compounds (4) was polymerized with isophorone diisocyanate at 70° C. without solvent using Thermomixer for 20 hr. The peaks shifts in FT-IR spectra were observed in FIG. 16 (B to D).

In FIG. 16, FT-IR spectra show the peak shifts of functional groups in each reaction step. (A) TMPCC: the strong peak in 1726 cm⁻¹ indicates carbonyl group of cyclic carbonate. (B) TMP diurethanes ring-opened by diamines: a new peak at 1675 cm⁻¹ indicates an amide (urethane) bond, polyurethanes from di-urethanes (B).

### Synthesis and Reactions of to Obtain Isocyanate Free Polyurethane Coating Compositions

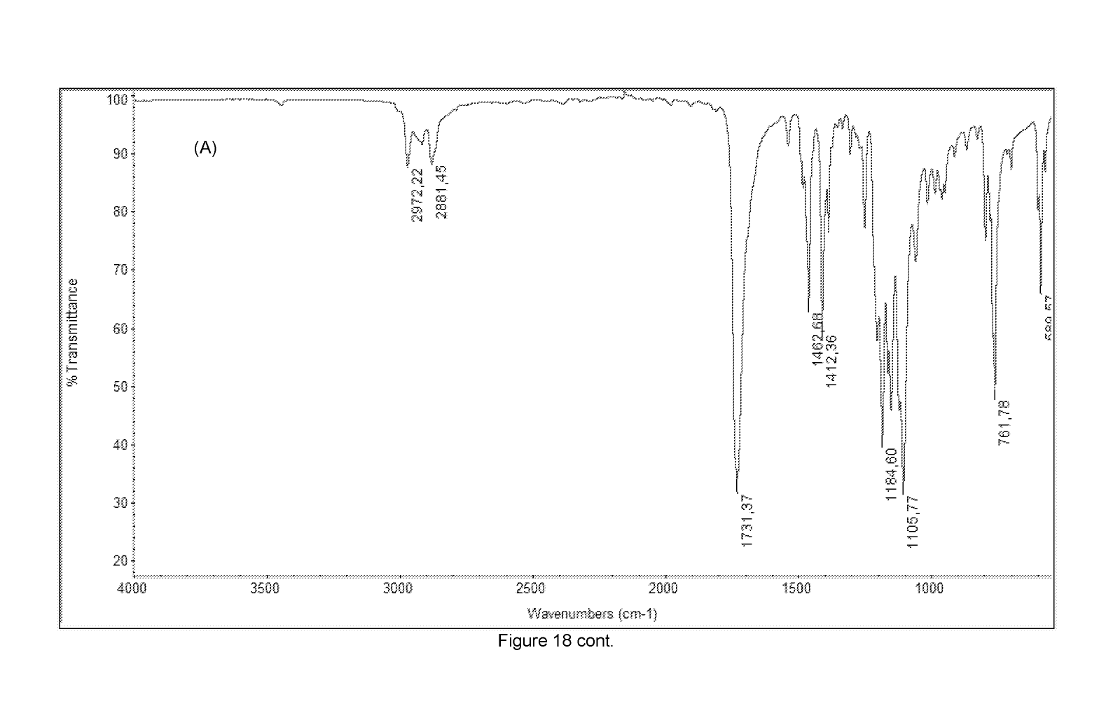

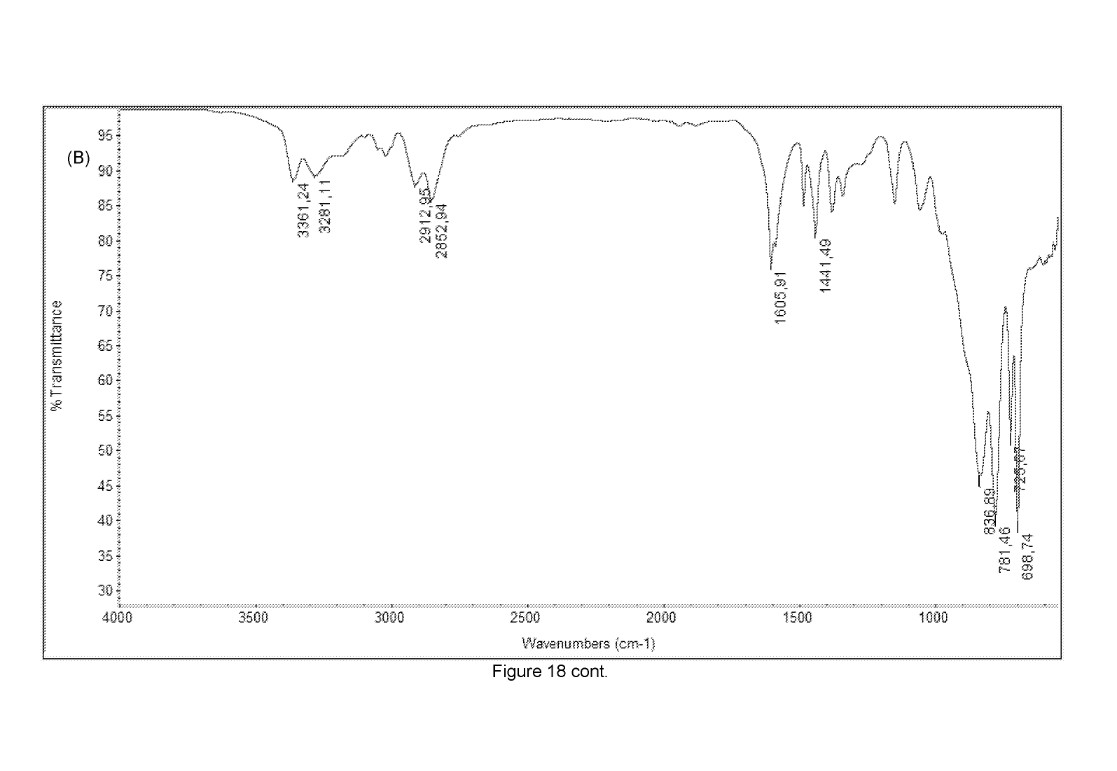

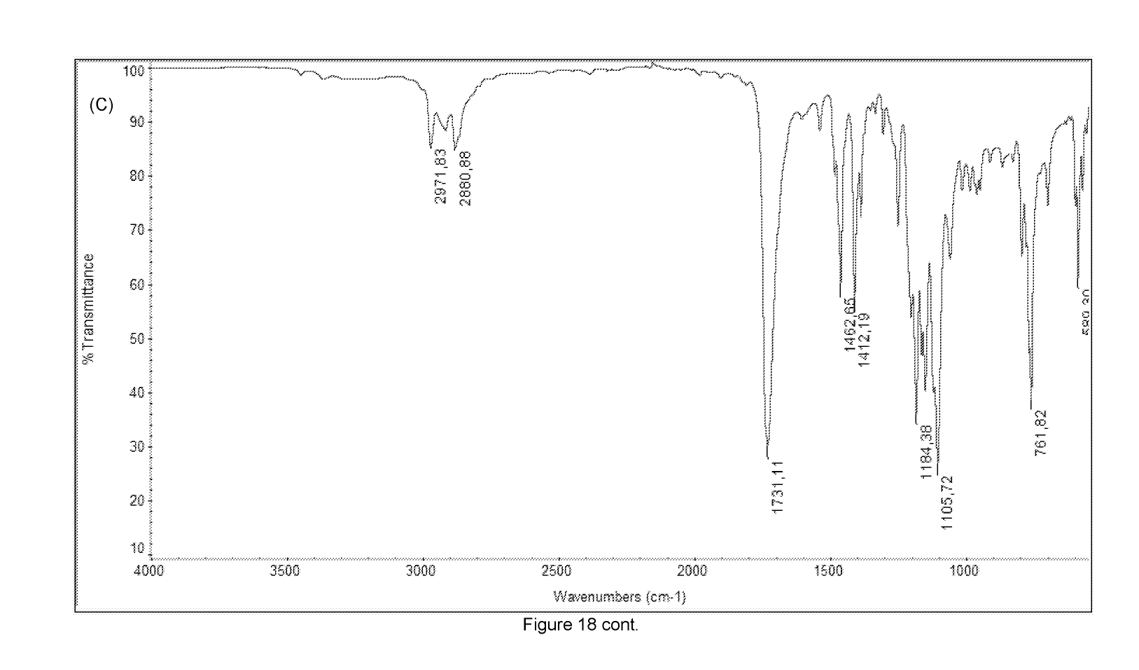

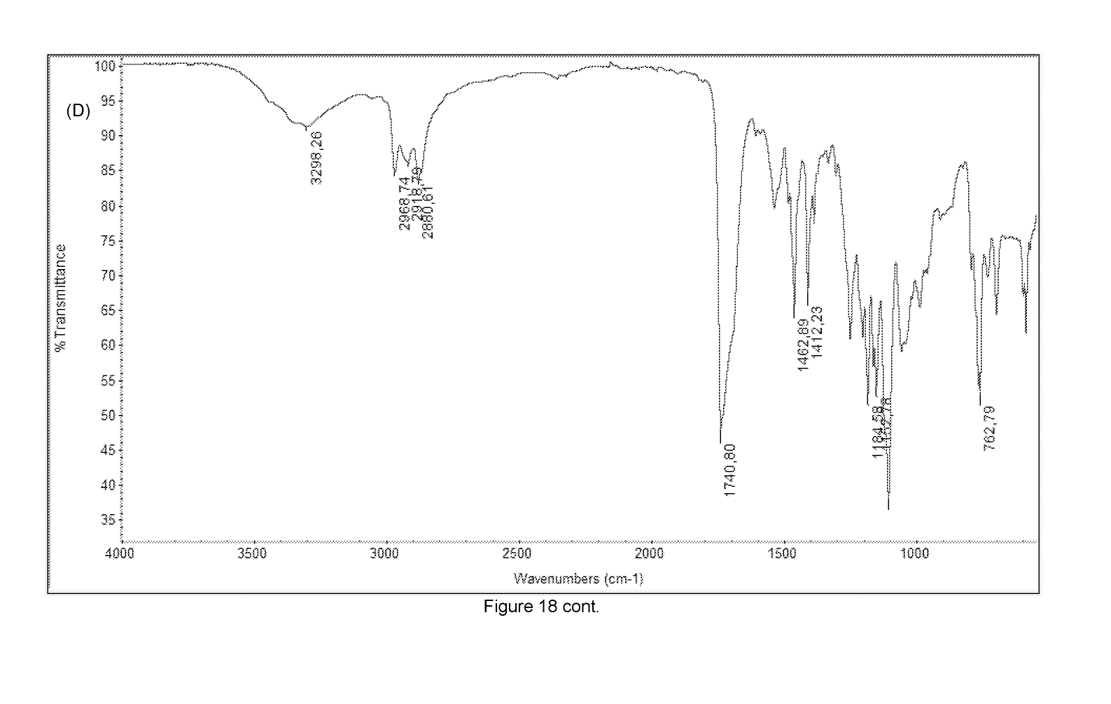

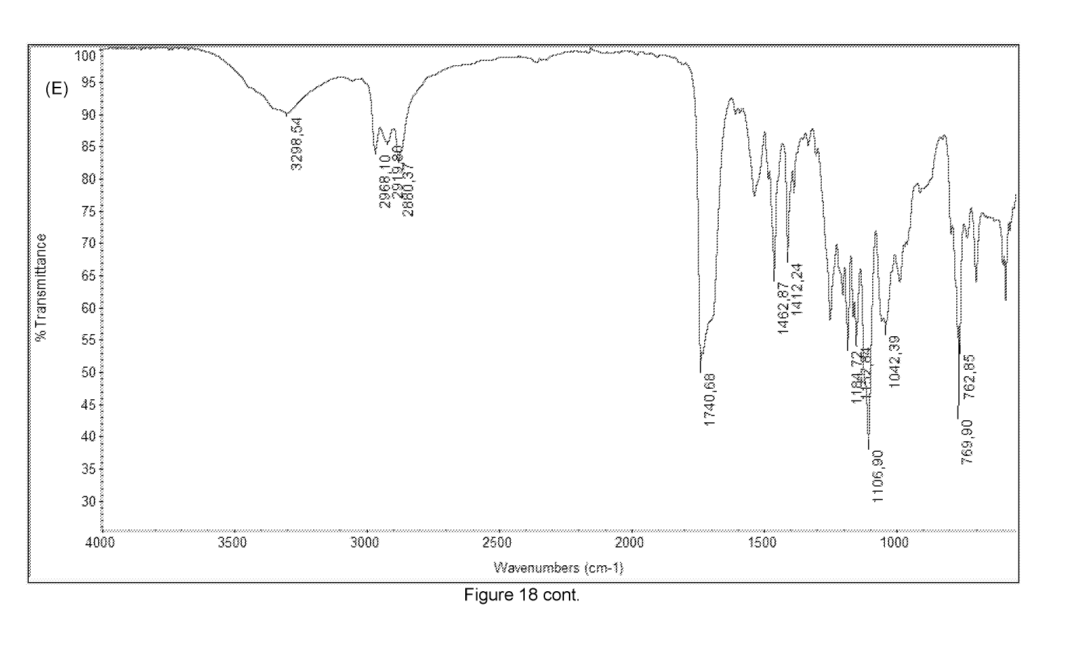

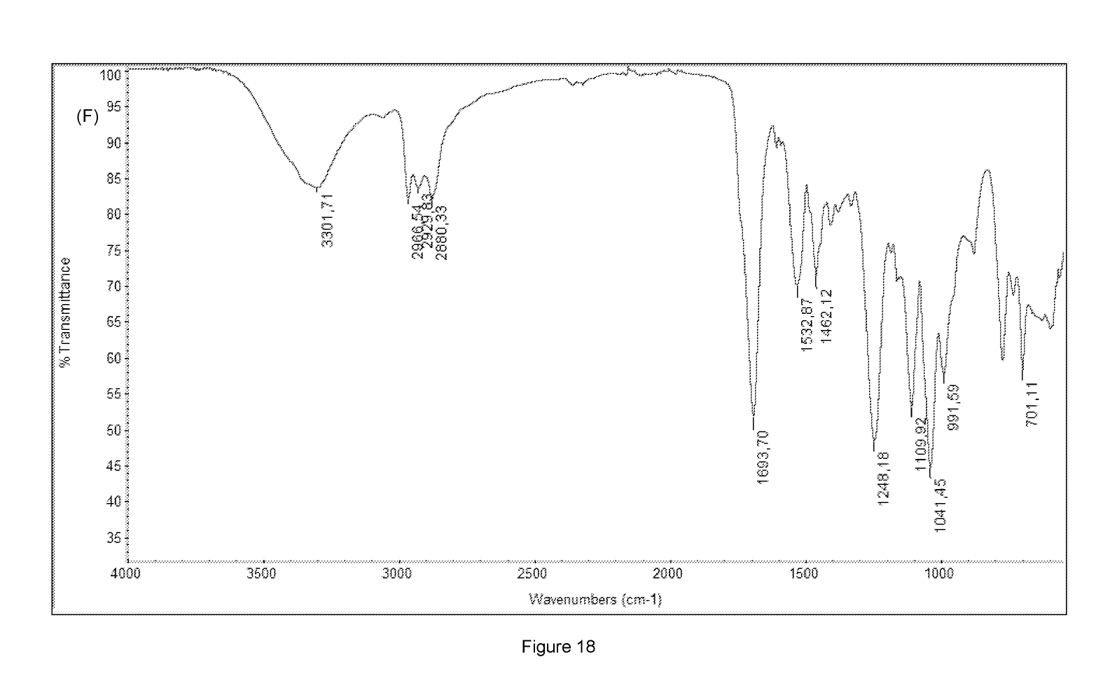

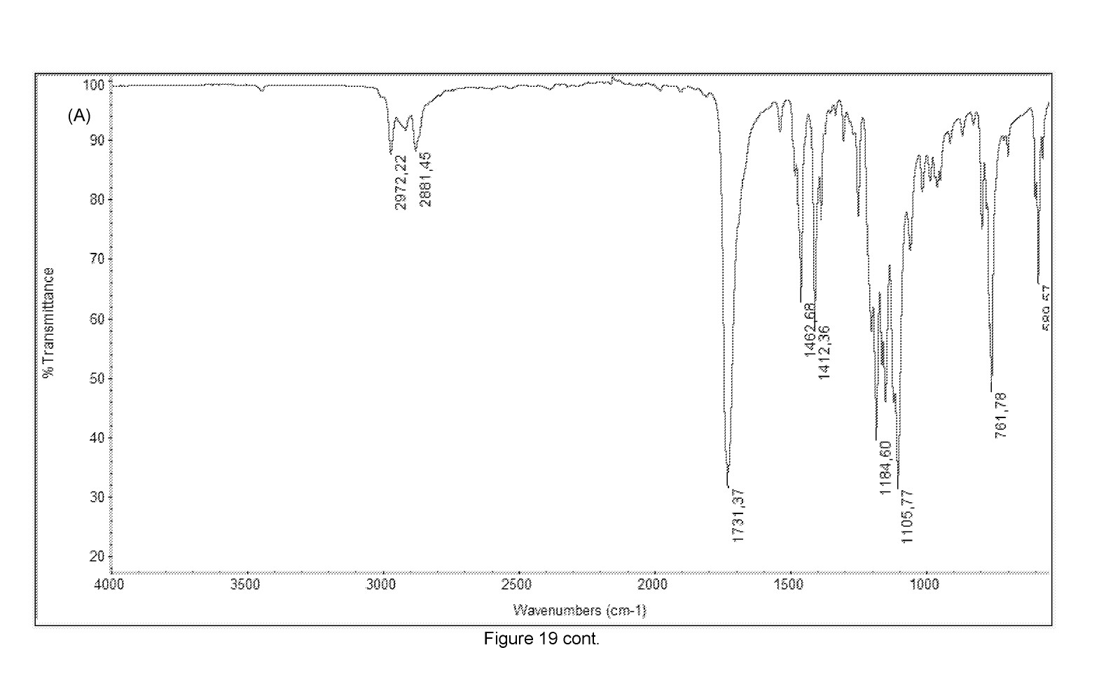

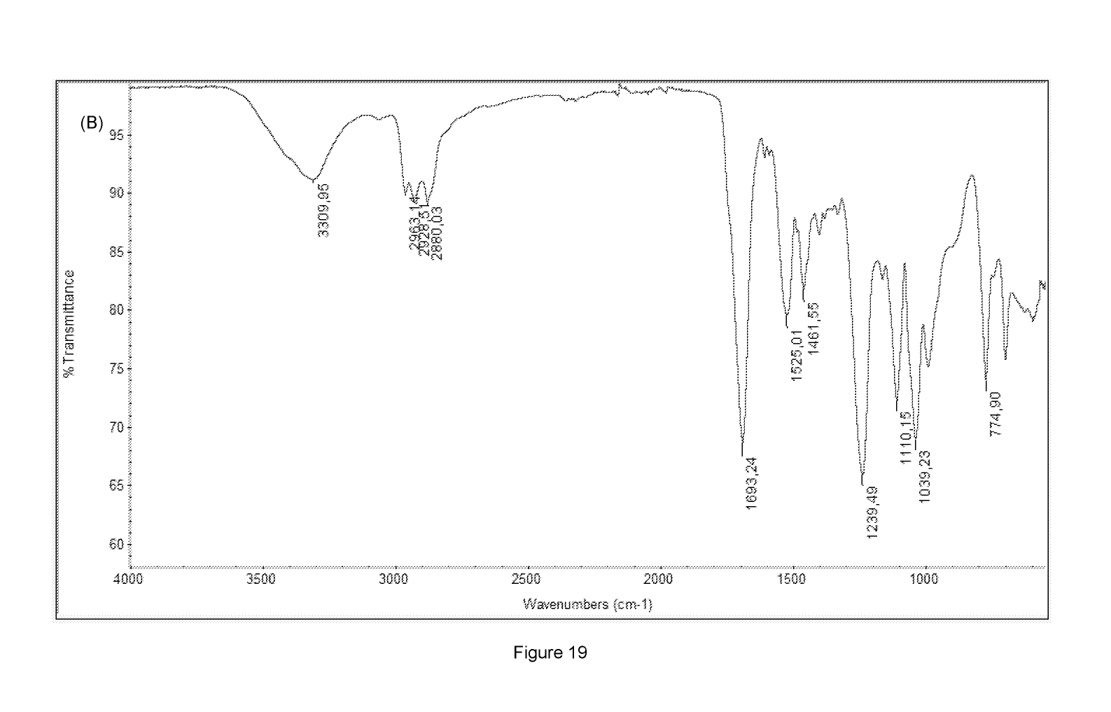

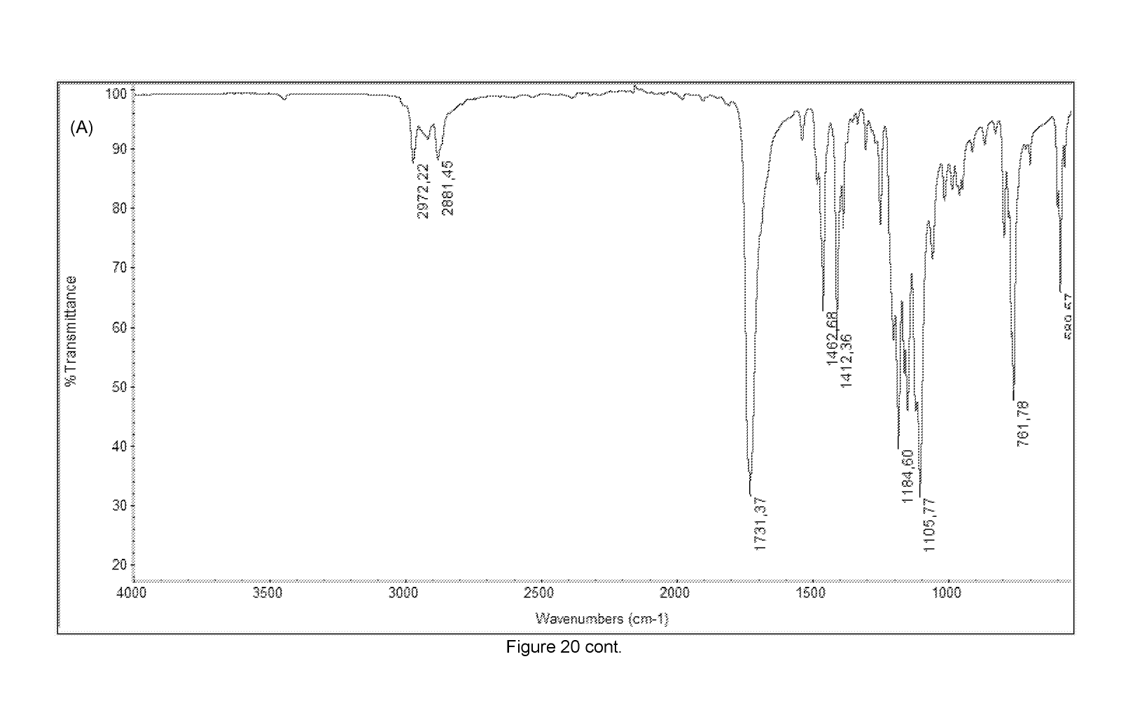

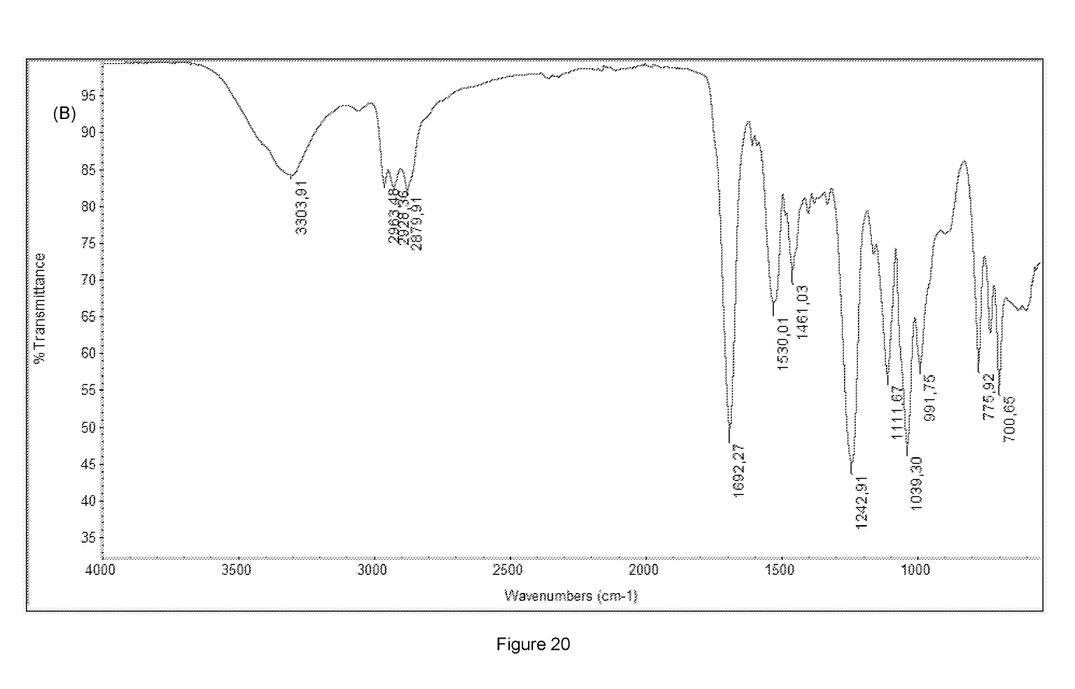

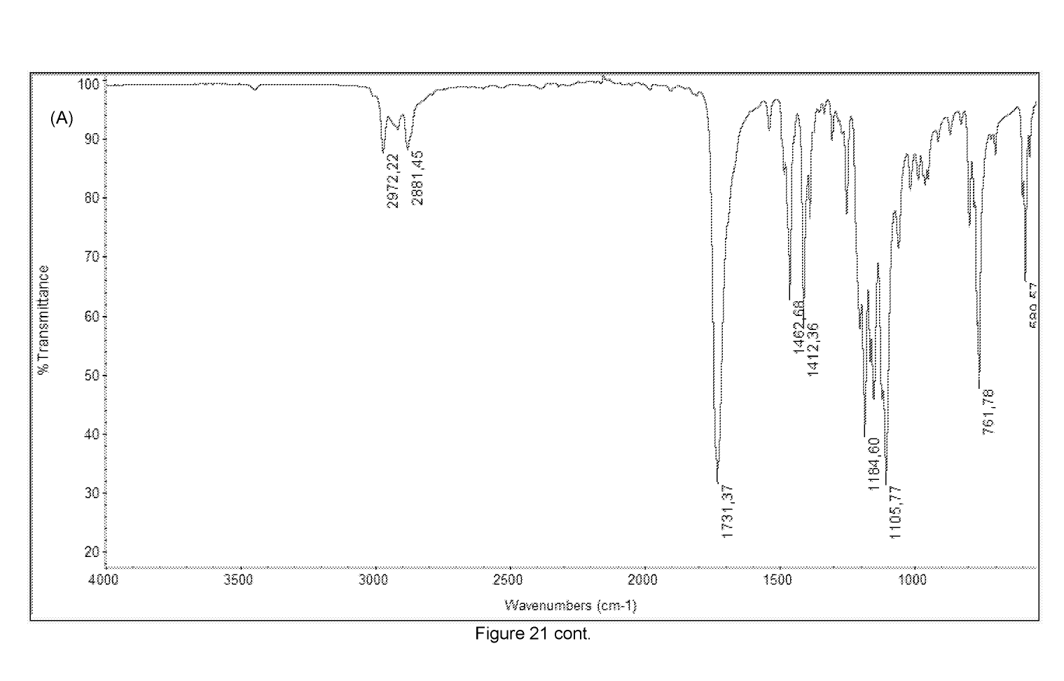

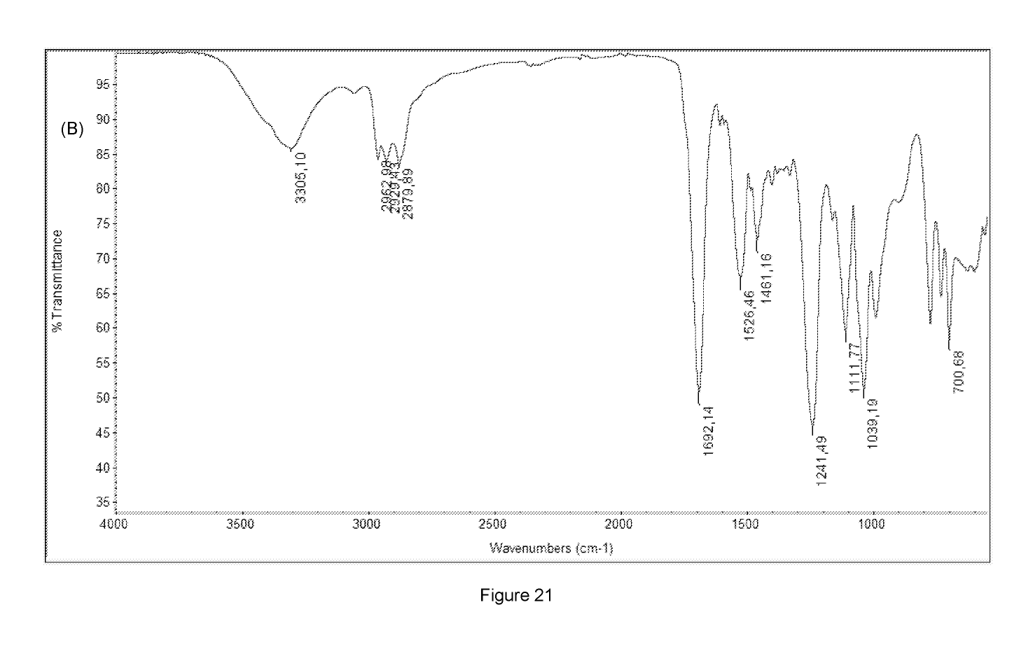

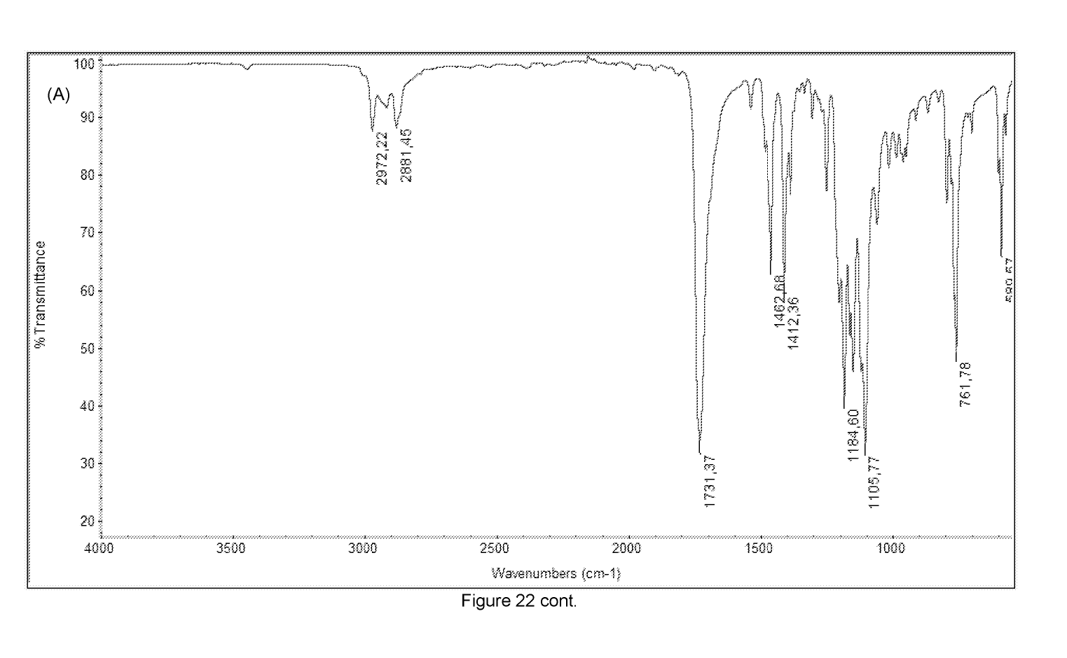

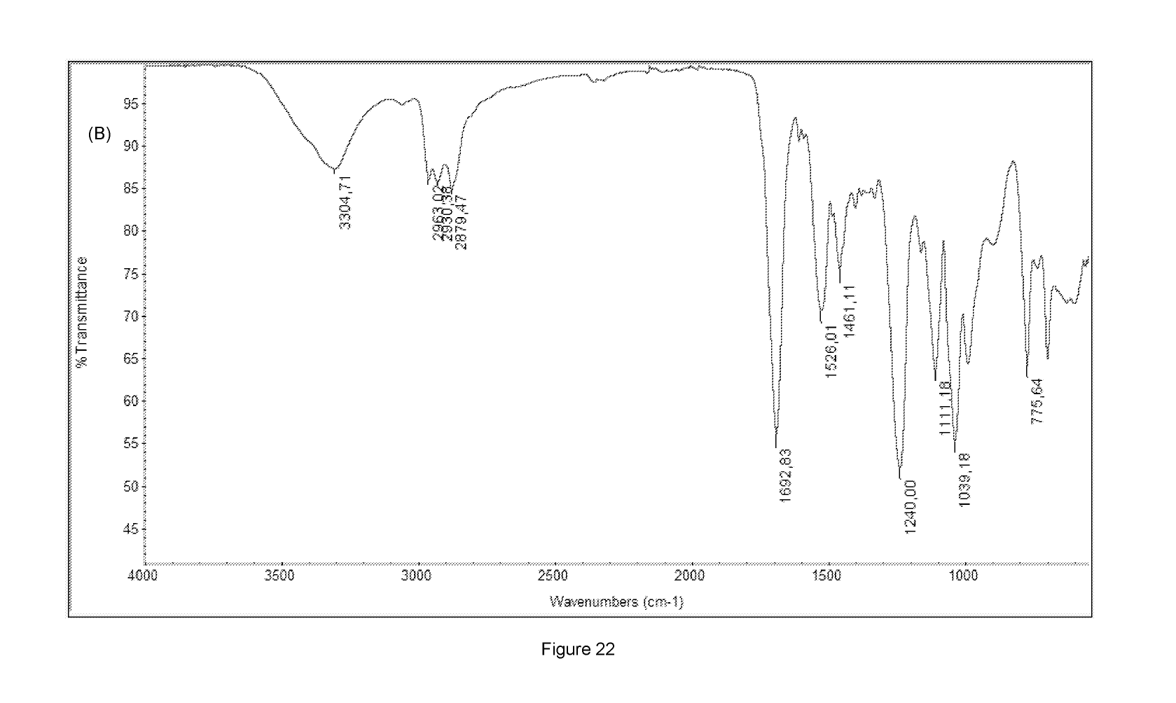

As a representative polymerization, the reaction of diTMPdiCC and XDA was performed in dichloromethane at RT without any additives and catalyst. In FIG. 18, FT-IR spectra show the peak shifts of functional groups in each reaction step at RT. (A) diTMPdiCC: the strong single peak in 1730 cm⁻¹ indicates carbonyl group of cyclic carbonate. (B) XDA: a multiple (broad) peak at 3361 and 3281 cm⁻¹ indicates primary amine. (C) Mixture of diTMPdiCC and XDA: the strong single peak in 1750 cm⁻¹ indicates carbonyl group of cyclic carbonate and weak peak around 3300 cm⁻¹ indicates primary amine of XDA. Peak intensity of XDA was weak compared to diTMPdiCC. (D) Homogenized solution of diTMPdiCC and XDA in dichloromethane: the strong peak in 1730 cm⁻¹ indicates carbonyl group of cyclic carbonate. The reaction takes place immediately in homogenized solution. Shoulder peak in 1690 cm⁻¹ indicates an amide bond of urethane group and a new peak at 3000-3500 cm⁻¹ appeared for —OH group resulting from ring opening of cyclic carbonate. (E) Homogenized solution of diTMPdiCC and XDA in dichloromethane after 1 min reaction: Shoulder peak in 1690 cm⁻¹ indicating an amide bond of urethane group and a new peak at 3000-3500 cm⁻¹ of —OH group resulting from ring opening of cyclic carbonate were stronger than those of time zero. (F) Homogenized solution of diTMPdiCC and XDA in dichloromethane after 5 min reaction: A shifted strong peak in 1690 cm⁻¹ indicates an amide bond of urethane group and a new strong peak at 3000-3500 cm⁻¹ appeared for —OH group resulted from ring opening of cyclic carbonate. FT-IR analyses were performed using Nicolet-iS5 (Thermo Scientific, USA).

### Example 10

### Coating from diTMPdiCC with Amines (1:1.1 Ratio) without Solvent

25 mg (0.083 mmol) diTMPdiCC was placed and heated at 90-110° C. on the glass. 1.1 molar ratio of diamine was added and homogenized. The melted and homogenized material was directly applied on the glass, and cured at 90° C. or 110° C. for certain time. The coating on the glass was cooled to RT and kept in the Lab. Hardness after 1 hr, 1 day and 2 days, and transparency after 1 hr were determined.

TABLE 5

| | | | | Hardness | | | |
|---|---|---|---|---|---|---|---|
| | | Curing | | after drying | | | |
| | | temp | Time | 1 | 1 | 2 | Transparency |
| Run | Diamine | (° C.) | (min) | hr | day | days | (0-5) |
| 1 | XDA | 110 | 5 | 2H | 2H | 2H | 4 |
| 2 | EDA | 110 | 5 | 2H | 2H | 2H | 4 |
| 3 | HMDA | 110 | 5 | 2H | 2H | 2H | 4 |
| 4 | IPDA | 110 | 5 | 2H | 2H | 2H | 4 |
| 5 | IPDA | 110 | 30 | 2H | 2H | 4H | 4 |
| 6 | XDA | 110 | 30 | 2H | 2H | 4H | 4 |
| 7 | ED600 | 110 | 30 | <4B | <4B | <4B | 5 |
| 8 | ED600 | 110 | 60 | <4B | <4B | <4B | 5 |
| 9 | XDA | 90 | 30 | 2H | 2H | 2H | 3 |
| 10 | HMDA | 90 | 30 | 2H | 2H | 2H | 2 |
| 11 | IPDA | 90 | 30 | HB | HB | 2H | 1 |

XDA (Xylylenediamine); EDA (Ethylenediamine); HMDA (hexamethylenediamine); IPDA (Isophoronediamine); ED600 (Jaffamine ED600).

### Example 11

### Coating from diTMPdiCC with Amines (Different Ratio) without Solvent

25 mg (0.083 mmol) diTMPdiCC was placed and heated at 110° C. on the glass. 1.5 or 0.67 molar ratio of diamine was added and homogenized. The melted and homogenized material was directly applied on the glass, and cured at 110° C. for 30 minutes. The coating on the glass was cooled to RT and kept in the Lab. Hardness after 1 h, 1 day and 2 days, and transparency after 1 h were determined.

TABLE 6

| | | | | Hardness | | | |
|---|---|---|---|---|---|---|---|
| | | Curing | | after drying | | | |
| | Ratio | temp | Time | 1 | 1 | 2 | Transparency |
| Run | (Diamine) | (° C.) | (min) | hr | day | days | (0-5) |
| 1 | 1.5 (XDA) | 110 | 30 | 2H | 2H | 2H | 4 |
| 2 | 1.5 (EDA) | 110 | 30 | 2H | 2-4H | 4H | 4 |
| 3 | 1 (XDA) | 110 | 30 | HB | HB | HB-2H | 4 |
| 4 | 1 (EDA) | 110 | 30 | HB | HB | HB-2H | 4 |

XDA (Xylylenediamine); EDA (Ethylenediamine).

### Example 12

### Coating from Crude diTMPdiCC with Amines (1:1.1 Ratio) without Solvent

25 mg (0.083 mmol, purity 82%) diTMPdiCC was placed and heated at 110° C. on the glass. 1.1 molar ratio of diamine was added and homogenized. The melted and homogenized material was directly applied on the glass, and cured at 110° C. for 30 minutes. The coating on the glass was cooled to RT and kept in the Lab. Hardness after 1 h, 1 day and 2 days, and transparency after 1 hr were determined.

TABLE 7

| | | Curing | | Hardeness after drying | | | |
|---|---|---|---|---|---|---|---|
| Run | Diamine | temp (° C.) | Time (min) | 1 hr | 1 day | 2 days | Transparency (0-5) |
| 1 | XDA | 110 | 30 | HB | 2H | 2H | 4 |
| 2 | EDA | 110 | 30 | HB | 2H | 2H | 4 |
| 3 | HMDA | 110 | 30 | 2H | 2H | 2H | 4 |
| 4 | IPDA | 110 | 30 | 2H | 2H | 4H | 4 |

XDA (Xylylenediamine); EDA (Ethylenediamine); HMDA (hexamethylenediamine); IPDA (Isophoronediamine).

### Example 13

### Coating from diTMPdiCC with Amines in Solvent by Curing at 110° C.

For using solvents such as acetonitrile, dichloromethane and dimethylformamide, 25 mg (0.083 mmol) diTMPdiCC was placed in 4 mL vial, to which was added 25 uL solvent and 1.1 molar ratio of a diamine at RT. After gently mixing for 2 minutes in RT, the solutions were applied on the glass surface, and cured at 110° C. for 5 minutes. For using solvents such as THF, 2-propanediol and ethanol, 25 mg (0.083 mmol) diTMPdiCC was placed in 4 mL vial, and added 25 uL solvent and 1.1 molar ratio of diamine at 60° C. After gently mixing for 5 minutes at 60° C., the solutions were applied on the glass surface, and cured at 110° C. for 5 or 30 minutes. The coating on the glass was cooled to RT and kept in the Lab. Hardness after 1 hr, 1 day and 2 days, and transparency after 1 hr were determined.

TABLE 8

| | | | Curing | | Hardness after drying | | | Transparency |
|---|---|---|---|---|---|---|---|---|
| Run | Diamine | Solvent | temp (° C.) | Time (min) | 1 hr | 1 day | 2 days | (0-5) |
| 1 | XDA | ACN | 110 | 5 | 2H | 2H | 2H | 4 |
| 2 | EDA | ACN | 110 | 5 | 2H | 2H | 2H | 5 |
| 3 | HMDA | ACN | 110 | 5 | 2H | 2H | 2H | 5 |
| 4 | IPDA | ACN | 110 | 5 | 2H | 2H | 4H | 5 |
| 5 | XDA | DCM | 110 | 5 | 2-4H | 2H | 4H | 5 |
| 6 | XDA | THF | 110 | 5 | 2-4H | 2H | 4H | 4 |
| 7 | XDA | 2PD | 110 | 5 | 2H | 2H | 2H | 4 |
| 8 | XDA | DMF | 110 | 5 | 2H | 2H | 2H | 5 |
| 9 | XDA | EtOH | 110 | 5 | 2H | 2H | 2H | 5 |
| 10 | EDA | 2PD | 110 | 5 | 2H | 2H | 4H | 4 |
| 11 | XDA | 2PD | 110 | 30 | 2H | 2H | 2H | 4 |
| 12 | EDA | 2PD | 110 | 30 | 2H | 2H | 4H | 4 |

XDA (Xylylenediamine); EDA (Ethylenediamine); HMDA (hexamethylenediamine); IPDA (Isophoronediamine); ACN (Acetonitrile); DCM (Dichloromethane); THF (Tetrahydrofuran); 2PD (2-propaneol); EtOH (Ethanol).

### Example 14

### Coating from diTMPdiCC with Amines in Solvent by Curing at 60° C.

For using solvents such as acetonitrile, dichloromethane and dimethylformamide, 25 mg (0.083 mmol) diTMPdiCC was placed in 4 mL vial, to which was added 25 uL solvent and 1.1 molar ratio of a diamine at RT. After gently mixing for 2 minutes in RT, the solutions were applied on the glass surface, and cured at 60° C. for 30 minutes. For using solvents such as THF and 2-propanediol, 25 mg (0.083 mmol) diTMPdiCC was placed in 4 mL vial, and added 25 uL solvent and 1.1 molar ratio of diamine at 60° C. After gently mixing for 5 minutes at 60° C., the solutions were applied on the glass surface, and cured at 60° C. for 30 minutes. The coating on the glass was cooled to RT and kept in the Lab. Hardness after 1 hr, 1 day and 2 days, and transparency after 1 h were determined.

TABLE 9

| | | | Curing | | Hardeness after drying | | | Transparency |
|---|---|---|---|---|---|---|---|---|
| Run | Diamine | Solvent | temp (° C.) | Time (min) | 1 hr | 1 day | 2 days | (0-5) |
| 1 | XDA | ACN | 60 | 30 | 2H | 2H | 4H | 4 |
| 2 | EDA | ACN | 60 | 30 | 2H | 4H | 4H | 4 |
| 3 | HMDA | ACN | 60 | 30 | HB | HB | HB | 5 |
| 4 | IPDA | ACN | 60 | 30 | HB | 2H | 2H | 5 |
| 5 | XDA | DCM | 60 | 30 | 2H | 2H | 2H | 5 |
| 6 | XDA | THF | 60 | 30 | 4H | 4H | 4H | 4 |
| 7 | XDA | 2PD | 60 | 30 | 2H | 2H | 2H | 4 |
| 8 | XDA | DMF | 60 | 30 | HB | HB | 2H | 2 |

XDA (Xylylenediamine); EDA (Ethylenediamine); HMDA (hexamethylenediamine); IPDA (Isophoronediamine); ACN (Acetonitrile); DCM (Dichloromethane); THF (Tetrahydrofurane); 2PD (2-propaneol).

### Example 15

### Coating from diTMPdiCC with Amines in Solvent by Curing at RT ° C.

For using solvents such as acetonitrile and dichloromethane, 25 mg (0.083 mmol) diTMPdiCC was placed in 4 mL vial, to which was added 25 uL solvent and 1.1 molar ratio of diamine at RT. After gently mixing for 10 minutes in RT, the solutions were applied on the glass surface, and cured at RT. The coating on the glass was kept at RT in the Lab. Hardness after 1 hr, 1 day and 2 days and transparency after 1 h were determined.

TABLE 10

| | | | Curing | Hardeness after drying | | | |
|---|---|---|---|---|---|---|---|
| Run | Diamine | Solvent | temp (° C.) | 1 hr | 1 day | 2 days | Transparency (0-5) |
| 1 | XDA | ACN | RT | HB | HB-2H | 2H | 5 |
| 2 | EDA | ACN | RT | HB | HB-2H | 2H | 5 |
| 3 | HMDA | ACN | RT | HB | HB-2H | 2H | 4 |
| 4 | IPDA | ACN | RT | HB | HB-2H | 2H | 1 |
| 5 | XDA | DCM | RT | 2H | 2H | 2H | 4 |

XDA (Xylylenediamine); EDA (Ethylenediamine); HMDA (hexamethylenediamine); IPDA (Isophoronediamine); ACN (Acetonitrile); DCM (Dichloromethane).

1. A method of producing a functionalized cyclic carbonate comprising the steps of:
a) providing a polyol having at least one functional group chosen from the group of hydroxyl, alkylhydroxyl, allyl, allylether, acryl, and methacryl;
b) admixing a dialkyl carbonate or a biphenyl carbonate and said functionalizes polyol of a) forming a mixture;
c) heating the mixture of b) to obtain a cyclic functionalized carbonate.

2. The method according to claim 1, wherein said polyol has a formula selected from 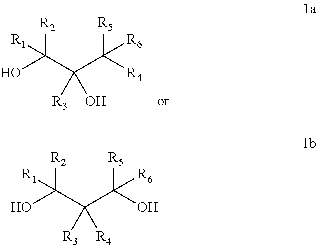
and the obtained obtained cyclic functionalized carbonate has a formula selected from 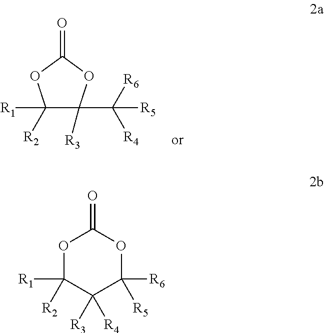
or their corresponding dimers.

3. The method according to claim 1 or 2, wherein said heating is performed at a temperature of at least 80° C., preferably at least 90° C., preferably at least 100° C., preferably at least 120° C., preferably at least 140° C.

4. The method according to claim 1, wherein the obtained functionalized cyclic carbonates were collected via a separation process.

5. The method according to claim 4, wherein the separation process is followed by a purification step.

6. The method of producing a functionalized monourethane and/or diurethane comprising the steps of:
i) providing a functionalized cyclic carbonate according to claim 1;
ii) providing at least one compound selected from the group alkylamine, aromatic amine and diamine;
iii) forming a mixture of said carbonate of i) and said at least one compound of ii);
iv) allowing reaction of the mixture of iii) by ring opening;
v) obtaining a functionalized monourethane and/or diurethane.

7. The method according to claim 6, wherein the alkylamine may be chosen from hexylamine, cyclohexylamine and dipropylamine.

8. The method according to claim 6, wherein the diamine may be chosen from alkyldiamines, preferably 1,6-hexamethylenediamine, 1,2-diethylenediamine and isophorone diamine.

9. The method according to claim 6, wherein the reaction by ring opening of step iv) is performed at a temperature of at least 20° C., preferably at least 50° C., preferably at least 100° C., preferably at least 120° C., preferably at least 140° C.

10. The method according to claim 6, wherein the reaction by ring opening of step j) is performed at a temperature of at most 0° C., preferably at most −10° C.

11. The method of producing a polyurethane comprising the steps of:
A) providing a functionalized urethane and/or diurethane according to claim 6;
B) reacting the a functionalized urethane and/or diurethane of A) in at least one step by UV and/or thermal reaction and/or isocyanate; and
C) obtaining a polyurethane.

12. The method according to claim 11, wherein the reaction of step B) additionally involves a thiol compound.

13. The method according to claim 12, wherein the thiol compound is chosen from polythiols, preferably the polythiol compound is chosen from dithiols, trithiols and tetrathiols.

14. The method according to claim 11, wherein an initiator is used in the reaction of step D).

15. The method according to claim 14, wherein the initiator is selected from the group azo compounds of azobisisobutyronitrile (AIBN) and 1,1′-azobis(cyclohexanecarbonitrile) (ABCN), and organic peroxides of di-ter-butyl peroxide and benzoyl peroxide.

16. The method according to claim 11, wherein when the reaction of step D) is performed using thermal energy the temperature is at least 20° C., preferably at least 90°, preferably at least 100° C., preferably at least 120°, or preferably at least 140° C.

17. The method of producing a polyurethane comprising the steps of:
m) providing a bicyclic carbonate having the formula 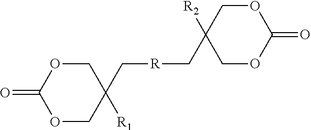
wherein R is chosen from a bond, oxygen, alkyl having 1-10 carbons, ketone, and ester;
R₁ and R₂ may independently be chosen from H, alkyl, hydroxyl, hydroxyalkyl, alkylcarbonyl, carbonylalkyl, alkoxycarbonyl, alkoxycarbonyloxy and carboxyl group;
and R3 is chosen from alkyl having 1-20 carbons, cycloalkyl, alkylphenyl, isophorone, polyamines, and derivatives thereof;
n) providing a diamine;
o) forming a mixture of said bicyclic carbonate of m) and said diamine of n);
p) allowing reaction of the mixture of o);
q) obtaining a polyurethane having the formula 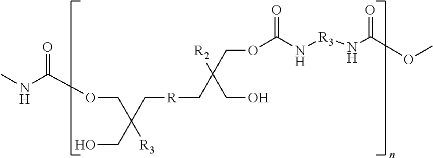
wherein R is chosen from a bond, oxygen, alkyl having 1-10 carbons, ketone, ester,
R₁, R₂ independently are chosen from H, alkyl, hydroxyl, hydroxyalkyl, alkylcarbonyl, carbonylalkyl, alkoxycarbonyl, alkoxycarbonyloxy and carboxyl group,
R3 is chosen from alkyl having 1-20 carbons, cycloalkyl, alkylphenyl, isophorone, polyamines and derivatives thereof.

18. The method of producing crosslinked polyurethanes or copolymers comprising the steps of:
I) providing a functionalized urethane and/or diurethane according to claim 6 or a polyurethane according to claim 11;
II) reacting the functionalized urethane and/or diurethane or polyurethane of I) by UV and/or thermal reaction or isocyanate;
III) obtaining a crosslinked polyurethane or copolymer product.

19. The method according to claim 18, wherein the reaction in step II) include a thiol compound.

20. The method according to claim 18, wherein the isocyanate compound is a polyisocyanate, preferably chosen from diisocyanate, preferably chosen from 1,6-hexamethylenediisocyanate, 1,2-diethylenediisocyanate, isophorone diisocyanate, and toluene-2,4-diisocyanate.

21. A cyclic carbonate comprising functional groups selected from the group hydroxyl, alkylhydroxyl, allyl, allylether, acryl, methacryl.

22. The cyclic carbonate obtained by the method according to claim 1.

23. The cyclic carbonate according to claim 21, wherein the cyclic carbonate is 5-membered or 6-membered cyclic carbonate, preferably 6-membered cyclic carbonate.

24. The cyclic carbonate according to claim 21, wherein the cyclic carbonate is monocyclic or multicyclic, preferably comprising 1 to 4 cyclic moieties, preferably comprising 1 to 3 cyclic moieties.

25. The cyclic carbonate according to claim 21 having a formula of 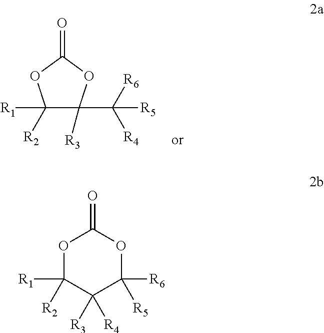
or their corresponding dimers
wherein R₁, R₂, R₃, R₄, R₅, R₆ independently are chosen from H, alkyl, hydroxyl, hydroxyalkyl, alkylcarbonyl, carbonylalkyl, alkoxycarbonyl, alkoxycarbonyloxy, carboxyl, allyl, acryl and methacryl, and
at least one of R₁, R₂, R₃, R₄, R₅ and R₆ is chosen from hydroxyl, hydroxyalkyl, allyl, allylether, acryl or methacryl group
or 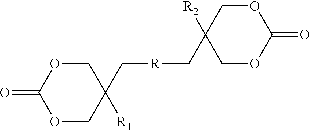
wherein R is chosen from a bond, oxygen, alkyl having 1-10 carbons, ketone, and ester;
R₁ and R₂ may independently be chosen from H, alkyl, hydroxyl, hydroxyalkyl, alkylcarbonyl, carbonylalkyl, alkoxycarbonyl, alkoxycarbonyloxy and carboxyl group;
and R3 is chosen from alkyl (1-20 carbons), cycloalkyl, alkylphenyl, isophorone, polyamines and derivatives thereof.

26. A functionalized monourethane and/or diurethane having a formula chosen from 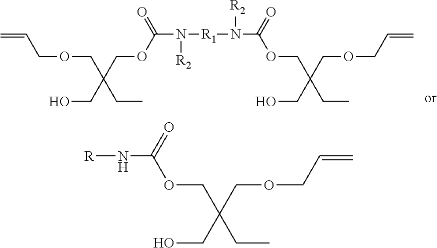
wherein R, R1, R2, R3, and R4 independently are chosen from H, hydroxyalkyl, alkyl, phenyl, hydroxyl, hydroxyalkyl, alkylcarbonyl, carbonylalkyl, alkoxycarbonyl, alkoxycarbonyloxy and carboxyl; 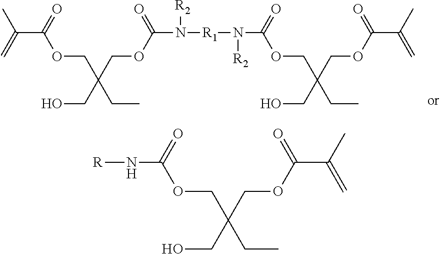
wherein R, R1, and R3 independently are chosen from H, alkyl, phenyl, hydroxyl, hydroxyalkyl, alkylcarbonyl, carbonylalkyl, alkoxycarbonyl, alkoxycarbonyloxy and carboxyl;
or 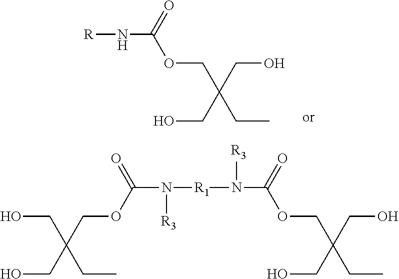
wherein R, R1, R2, and R3 independently are chosen from H, alkyl, phenyl, hydroxyl, hydroxyalkyl, alkylcarbonyl, carbonylalkyl, alkoxycarbonyl, alkoxycarbonyloxy and carboxyl group.

27. The functionalized monourethane and/or diurethane obtained by the method according to claim 6.

28. A polyurethane having a formula chosen from 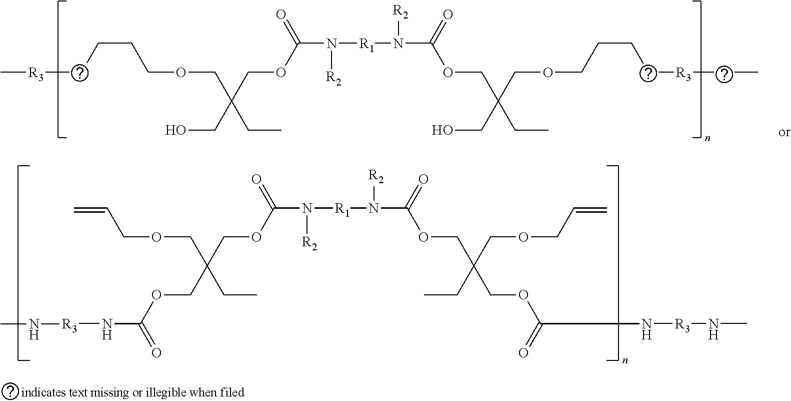
wherein R, R1, R2, R3, and R4 independently are chosen from H, hydroxyalkyl, alkyl, phenyl, hydroxyl, hydroxyalkyl, alkylcarbonyl, carbonylalkyl, alkoxycarbonyl, alkoxycarbonyloxy and carboxyl; 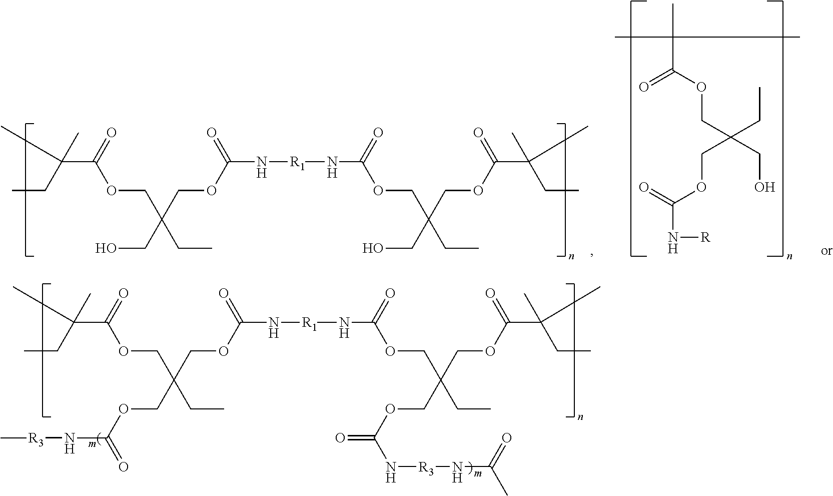
wherein R, R1, and R3 independently are chosen from H, alkyl, phenyl, hydroxyl, hydroxyalkyl, alkylcarbonyl, carbonylalkyl, alkoxycarbonyl, alkoxycarbonyloxy and carboxyl; 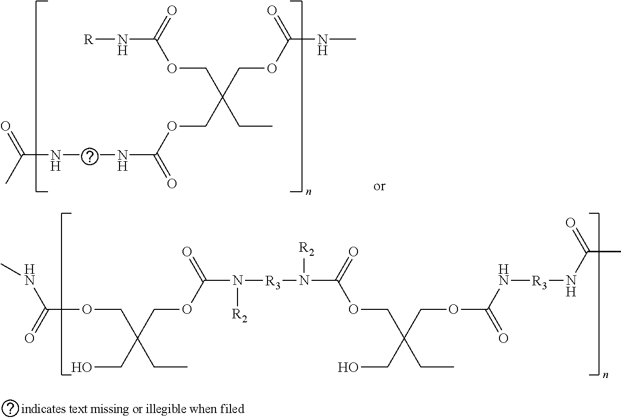
wherein R, R1, R2, and R3 independently are chosen from H, alkyl, phenyl, hydroxyl, hydroxyalkyl, alkylcarbonyl, carbonylalkyl, alkoxycarbonyl, alkoxycarbonyloxy and carboxyl;
or 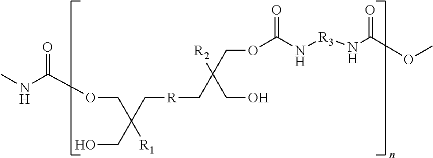
wherein R is chosen from a bond, oxygen, alkyl having 1-10 carbons, ketone, ester,
R₁, R₂ independently are chosen from H, alkyl, hydroxyl, hydroxyalkyl, alkylcarbonyl, carbonylalkyl, alkoxycarbonyl, alkoxycarbonyloxy and carboxyl group,
R3 is chosen from alkyl having 1-20 carbons, cycloalkyl, alkylphenyl, isophorone, polyamines and derivatives thereof.

29. The polyurethane obtained by the method according to claim 11.

30. The use of a functionalized urethane and/or diurethane according to claim 6 or a polyurethane according to claim 11 for the production of foams, seatings, seals, sealants, coatings or adhesives.

31. The use according to claim 30, for the production of insulation foams, packaging foames, structural foam, high resiliency foam, footwear soles, simulated wood, integral skin foam for vehicle interiors, facia and other exterior parts of a vehicle, durable elastomeric wheels and tires, synthetic fibers, print rollers, cast elastomers, reaction injection molded plastic, material enclosing electronic components or implants and devices of medical use.
